(19) United States
(12) Patent Application Publication
(10) Pub. No.: US 2022/0395181 A1
(43) Pub. Date: Dec. 15, 2022

(54) SYSTEM AND METHODS FOR SENSOR-BASED DETECTION OF SLEEP CHARACTERISTICS AND GENERATING ANIMATION DEPICTION OF THE SAME

(71) Applicant: Wesper Inc., New York, NY (US)

(72) Inventors: Amir REUVENY, New York, NY (US); Ahud MORDECHAI, Petah-Tikva (IL); Mordechai PERLMAN, Cresskill, NJ (US); Nathan Harold BENNETT, Somerville, MA (US)

(21) Appl. No.: 17/840,933

(22) Filed: Jun. 15, 2022

Related U.S. Application Data
(60) Provisional application No. 63/210,668, filed on Jun. 15, 2021.

(51) Int. Cl. A61B 5/00; A61B 5/1455; A61B 5/11; A61B 5/024; A61B 5/01

(52) U.S. Cl. CPC A61B 5/0024; A61B 5/4809; A61B 5/4812; A61B 5/6833; A61B 5/14552; A61B 5/7425; A61B 5/7435; A61B 5/1126; A61B 5/02405; A61B 5/01; A61B 2562/0247; A61B 2562/0219

(57) ABSTRACT

A system for monitoring a sleep of a user includes a plurality of patches for placement adjacent to a surface of a body of a user, a processor, and a data communication system. Each patch from the plurality of patches includes at least one sensor. The data communication system transmits positional data generated by the plurality of sensors, including orientation data and motion data, to the processor. The processing of the positional data includes detecting a change in position of the body of the user between a first position and a second position. Based on the first image and the second image, an animation of a movement of the body from the first position to the second position is generated.

### CROSS-REFERENCE TO RELATED APPLICATIONS

This application claims priority to and the benefit of U.S. Provisional Patent Application No. 63/210,668, filed on Jun. 15, 2021, the disclosure of which is hereby incorporated by reference in its entirety.

### TECHNICAL FIELD

The present disclosure relates generally to systems, apparatus, and methods for monitoring a sleep parameter of a user, and more particularly to sensor-based detection and monitoring of sleeping positions in a home setting.

### BACKGROUND

Millions of people suffer from various forms of chronic sleep disorders (CSDs), including insomnia, sleep apnea, and periodic limb movement disorder (PLMD). CSDs may account for billions of dollars of lost work productivity. For example, sleep apnea alone has been estimated to cost workplaces $150 billion annually.

While the number of patients seeking help for CSDs has grown in recent years, a majority of those suffering from a CSD remain undiagnosed. A significant factor that disincentives potential patients from seeking help is the high cost. Professional assessments of sleep, such as administering a polysomnogram, usually engage a patient to spend a night at a “sleep lab” to monitor various factors while the patient is sleeping, such as brain activity, eye movements, heart rate, and blood pressure. These assessments typically involve expensive equipment and can cost upwards of $5,000 per night.

While home sleep tests designed to be self-administered by patients do exist, many such tests still use elaborate equipment that is assembled by the users (e.g., home assembly), which can be frustrating, and such equipment can be uncomfortable to wear. Many home sleep tests also attach multiple parts to a patient's body, including an oxygen monitor, nasal tubes, and chest straps. Additionally, these tests are often inaccurate. Therefore, multiple attempts are usually conducted to capture meaningful data. Furthermore, the recorded data in these tests is often sent to physicians for analysis, thereby adding a logistical obstacle to the diagnosis and monitoring of a potential CSD.

### SUMMARY

In some embodiments, a system for monitoring a sleep of a user includes a plurality of patches for placement adjacent to a surface of a body of a user, a processor, and a data communication system. Each patch from the plurality of patches includes at least one sensor. The data communication system transmits positional data generated by the plurality of sensors, including orientation data and motion data, to the processor. The processing of the positional data includes determining a first position of the body of the user at a first time and a first image based on the first position of the body of the user at the first time. A change in position of the body of the user is detected based on a measure function and a threshold value. In response to detecting the change in position of the body of the user, a second position of the body of the user is determined, at a second time subsequent to the first time, and a second image is determined based on the second position of the body of the user at the second time. Based on the first image and the second image, an animation of a movement of the body from the first position to the second position is generated.

In some embodiments, a method for monitoring a sleep of a user includes positioning a plurality of patches adjacent to a surface of a body of a user. Each patch from the plurality of patches includes an associated sensor from a plurality of sensors. The method also includes causing positional data generated by the plurality of sensors to be transmitted to a processor, the positional data including orientation data and motion data. The processing of the positional data via the processor includes determining a first position of the body of the user at a first time, determining a first image based on the first position of the body of the user at the first time, and detecting a change in position of the body of the user based on a measure function and a threshold value. In response to detecting the change in position of the body of the user, a second position of the body of the user at a second time subsequent to the first time is determined. A second image is determined based on the second position of the body of the user at the second time. Based on the first image and the second image, an animation is generated of a movement of the body from the first position of the body of the user at the first time to the second position of the body of the user at the second time.

In some embodiments, a non-transitory computer readable medium stores instructions that, when executed by a processor, cause the processor to perform operations including determining a first position of the body of the user at a first time, determining a first image based on the first position of the body of the user at the first time, and detecting a change in position of the body of the user based on a measure function and a threshold value. The operations also include, in response to detecting the change in position of the body of the user, determining a second position of the body of the user at a second time subsequent to the first time, and determining a second image based on the second position of the body of the user at the second time. An animation of a movement of the body from the first position of the body of the user at the first time to the second position of the body of the user at the second time is generated based on the first image and the second image.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 is an example system for obtaining sleep data during a sleep of a user, according to some embodiments.

FIG. 2A is a first example patch for obtaining sleep data during a sleep of a user, according to some embodiments.

FIG. 2B is a second example patch for obtaining sleep data during a sleep of a user, according to some embodiments.

FIG. 3A are example parameters that may be collected during a sleep of a user, according to some embodiments.

FIG. 3B are example motions of a body of a user during a sleep of a user, according to some embodiments.

FIG. 4A is an example diagram for collecting and processing data obtained during a sleep of a user, according to some embodiments.

FIG. 4B is another example diagram for collecting and processing data obtained during a sleep of a user, according to some embodiments.

FIGS. 5A-5C are example interfaces for displaying and interacting with information describing sleep characteristics of a user, according to some embodiments.

FIG. 6 is an analysis module for differentiating body positions of a user, according to some embodiments.

FIGS. 7A-7B are example processes for generating an animation, according to some embodiments.

FIGS. 8A-8E are examples of insights reported via an interface of a compute device, according to some embodiments.

FIG. 9 is a diagram of obtaining, transmitting and processing data, according to some embodiments.

FIG. 10A is an example printed circuit board for a patch, according to some embodiments.

FIG. 10B is an example implementation of a patch, according to some embodiments.

FIG. 11 shows a mechanical model for optimizing the placement of patches, according to some embodiments.

FIG. 12 is an example process for optimizing the placement of patches, according to some embodiments.

FIG. 13 includes example graphs showing sleeping trends according to some embodiments.

FIG. 14 includes example graphs showing a correlation between a number of breathing events per hour and a number of hours slept during a night according to some embodiments.

### DETAILED DESCRIPTION

The present disclosure describes systems, apparatuses, and methods for monitoring various characteristics of a sleep of a user, and more particularly to detection, monitoring, and graphical depiction of sleeping positions in a home setting based on sleep data obtained using one or more flexible elements. In some embodiments, the one or more flexible elements are conductive and/or are configured to exhibit modified electrical properties in response to an applied force.

The present disclosure addresses various challenges associated with monitoring a sleep of a person without using elaborate and uncomfortable equipment, such as nasal tubes, and chest straps. Further, to address challenges associated with inaccuracies associated with recorded sleep data, apparatuses, systems, and methods described herein employ patches with multiple sensors to monitor sleep parameters, such as respiratory effort, of a user. Using multiple sensors allows for accurate sleep data recording.

In various embodiments, a patch may be configured to conform to a surface of the user (or the user's clothes). In an example embodiment, a sensor of a patch may include a flexible element that is coupled to the patch and includes a conductive material, such as a conductive, nonwoven fabric or other textile and/or a conductive polymer. In some cases, the patch may include a power source electrically coupled to the flexible element and an electrical circuit electrically coupled to the power source and the flexible element. The electrical circuit is configured to detect, during use, a change in an electrical property of the flexible element. The electrical property of the flexible element can include, for example, resistance, reactance, impedance, or any other suitable property.

Additionally, or alternatively, the patch may use an antenna to receive energy via radio-frequency electromagnetic waves from an external device and use the received energy to supply power to one or more internal electrical components of the patch. Using such a configuration, the patch may not be required to have a discrete onboard power source (e.g., a battery) and may, thus, have a smaller size. In some cases, a patch may be powered by person's metabolic processes (e.g., a heat emitted by a person, or a sweat of a person's skin).

In an example embodiment, a patch can be attached to the skin of the user (e.g., on the torso of the user) while the user is sleeping. Breathing of the user can cause the skin to compress or stretch, thereby compressing and stretching the flexible element accordingly. The compression and stretching of the flexible element, in turn, changes its electrical property, which can be measured by the electrical circuit. In this manner, the breathing of the user can be monitored by monitoring the electrical property of the element.

In some embodiments, devices (e.g., respiratory monitors, sleep monitors, sleep disorder detectors, etc.) based on the approach described herein can be configured as a patch that can be conveniently worn by the user or attached to the user without causing excessive discomfort to the user. Therefore, the breathing and/or sleep of the user can be readily monitored in a home setting.

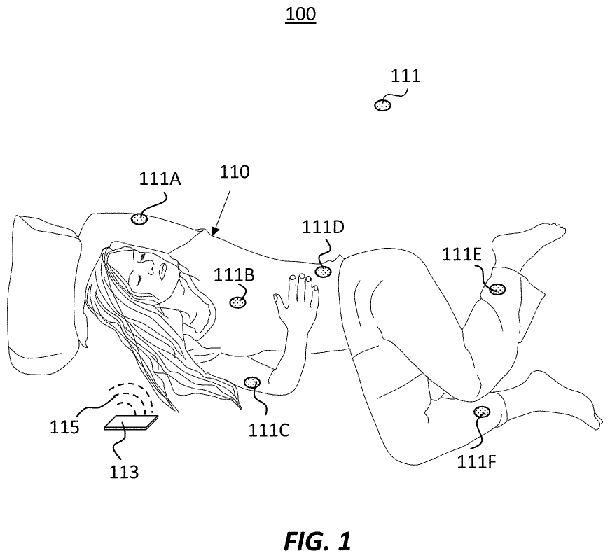

FIG. 1 shows an example of a system 100 for monitoring a sleep of a user 110 sleeping in a back-side position (i.e., the position characterized by a user laying predominantly on her back and slightly on her side). System 100 includes multiple patches (patches 111A-111F, as shown in FIG. 1), with each patch having at least one associated sensor. In an example embodiment, each patch is configured to be positioned adjacent to a surface of a body of user 110. As described herein, a patch at any position adjacent to a body of user 110 is referred to as patch 111, while patches at specific positions are referred by their corresponding numbers 111A-111F. In an example embodiment, patch 111 may be positioned adjacent to a body of user 110 using any suitable means. For example, patch 111 may be adhered to skin of user 110, adhered or otherwise attached to clothes of user 110, magnetically attached to a metallic tag adjacent to user 110's body (e.g., the metallic tag may be adhered to user 110's clothes), clipped to user 110's clothes, or attached via any other suitable means (e.g., bracelets, belts, chains, and the like) to user 110's body.

In an example embodiment, patches 111A-111F may be configured to be the same (i.e., have the same sensors). Alternatively, one patch (e.g., patch 111B) may have a first set of sensors, and another patch (e.g., patch 111E) may have a second set of sensors, with at least one sensor in the second set of sensors being different from sensors in the first set of sensors. In some cases, patch 111B may include more sensors than patch 111E. For example, patch 111B may have a sensor for detecting a motion of user 110's chest, while patch 111E may not contain such a sensor. Patch 111E may include a pulse measuring sensor, while patch 111B may include a temperature sensor. In some implementations, patches 111A-111F may be single-use patches, and in other implementations, patches 111A-111F may be multiple use patches. In some implementations, patches 111A-111F may have internal power supplies (also referred to herein as power sources) which may be rechargeable for example via wireless or contact charging.

As described above, each patch 111 can include one or more sensors for detecting motions and orientations of the user 110's body. For example, a patch 111 may include one or more accelerometer sensors, gyroscope sensors, level measuring sensors, geomagnetic sensors, proximity sensors, pressure sensors, and the like. In an example embodiment, a single-axis accelerometer and a multi-axis accelerometer (or a plurality of such accelerometers) can be used to detect both the magnitude and the direction of a proper acceleration (herein, the proper acceleration is the acceleration (the rate of change of velocity) of a body in its own instantaneous rest frame, e.g., the resting body will measure an acceleration due to Earth's gravity of g≈9.81 m/s²), as a vector quantity, and can be used to sense an orientation of a body of user 110, coordinate accelerations, vibrations, shocks, and falling in a resistive medium. Any suitable design of sensors may be used (e.g., sensors may be micro-electromechanical (MEMS) devices, and may include electrical, piezoelectric, optical, piezoresistive, and/or capacitive components). Thus, one or more accelerometers may be used to detect both motions and orientations of user 110's body. For instance, an accelerometer may detect whether user 110 is standing or laying down, while several accelerometers, placed in appropriate positions over user's body may determine more complex body positions (e.g., whether a user is sitting or reclining). In an example embodiment, system 100 may be configured to determine, based on data received from accelerometers, whether the user is in a vertical position, a seated position, a reclined position, or a horizontal position.

In an example embodiment, data acquired by an accelerometer can be used to determine a respiratory effort of user 110. In some embodiments, accelerometer data can be analyzed in combination with data from other sensors (whether on the same patch or on a different patch within a common system) to investigate the respiratory efforts of the user in different sleep positions and/or to improve signal/data quality. In some embodiments, the signal processing associated with respiratory effort can be based on the accelerometer data. Such investigation may help identify the possible sleep disorders of the user in certain particular positions.

Additionally, patches 111A-111F may measure various other parameters associated with a user (e.g., user 110) during her sleep. For example, a patch 111 may include any one of (or any combination of): a pressure sensor, a sensor for detecting breathing, a pulse sensor, an oximeter, a humidity sensor, a temperature sensor a vibrational sensor, an audio sensor (e.g., a microphone), a nasal pressure sensor, a surface airflow sensor, a proximity sensor, a camera, a reflectometer, or a photodiode. Additionally, one of (or a plurality) of patches, as well as other sensors of system 100 (as discussed below), may measure environmental parameters such as temperature and humidity of an environment (e.g., a room) in which user 110 is located, lighting levels in the room, audio levels within the room, an airflow within the room, and the like. In an example embodiment, a first temperature sensor may measure a temperature of user 110's body, and a second temperature sensor may measure a temperature in the room. Similarly, one humidity sensor may measure a humidity of user 110's skin (such measurements may be done, for example, by measuring a skin resistance), and another humidity sensor may measure a humidity of air in the room.

In an example embodiment, a pressure sensor may be configured to measure a pressure exerted on a surface of patch 111. For example, a pressure sensor may measure a higher pressure when a weight of a person (e.g., user 110) is located above patch 111 (i.e., patch 111 is located between a bed's surface and user 110's body). Alternatively, pressure sensors of patches 111A-111F may not record significant pressure values as they are not located between the bed's surface and user 110's body.

Patch 111 may include a pulse sensor, such as, for example, a pulse oximeter. For such a configuration, the pulse oximeter may be a combination of a pulse and oximeter sensor. The pulse oximeter is configured to measure the oxygen saturation level (e.g., SpO2) and a heart rate of user 110. As used herein, the SpO2 of a user refers to the percentage of oxygenated hemoglobin (i.e., hemoglobin that contains oxygen) compared to the total amount of hemoglobin (i.e., the total amount of oxygenated and non-oxygenated hemoglobin) in the blood of the user.

In some embodiments, the pulse oximeter can measure the SpO2 of the user via an optical method. Using such a method, the pulse oximeter employs an emitter, such as a laser or a light emitting diode (LED) to emit a light beam (usually red or near infrared) to the skin of the user. A detector in the pulse oximeter is configured to detect light reflected, transmitted, or scattered from skin of the user. The SpO2 of the user can be derived from the absorption and/or reflection of the light beam. If the pulse oximeter determines that user 110's oxygen levels are below the normal range (e.g., below 95%), an alarm can be generated by an alarm device of system 100 to alert user 110. Further, the pulse oximeter may be configured to determine that user 110's heart rate is within an expected, predefined heart rate range (e.g., the expected range for the heart rate may be calibrated for user 110, and may be, for example, in a range of 50 to 100 beats per minute). In some cases, when the heart rate is outside the expected heart rate range, an alarm can be generated by an alarm device of system 100 to alert user 110. The alarm can be implemented as an audible sound, a visible indication (e.g., a flashing light), and/or a haptic feedback (e.g., a vibration, optionally at a predetermined frequency or with a predetermined periodicity or intensity).

In an example embodiment, patch 111 may include a first microphone sensor configured to capture sound near or surrounding user 110. In some embodiments, the microphone is configured to capture ambient noise. The ambient noise can include sound from user 110's breathing and/or snoring. This microphone data can be used, for example, to analyze the sleep quality of user 110. For example, the sound from user 110's breathing can be used to analyze the breath rhythm of the user, which in turn can indicate the sleep quality. The sound from the snoring of user 110 can also reveal the sleep quality. For example, detection of excess snoring may be correlated with a high risk of sleep disorder.

In some embodiments, patch 111 may include a second microphone sensor configured to capture sound from the heart, lungs, or other organs (e.g., wheezes, crackles, or lack thereof) of user 110. In some embodiments, system 100 may include a suitable data processing device (as further described below) to identify and/or distinguish sounds from different sensors so as to improve the accuracy of subsequent analysis. Such identification can be based on, for example, the rhythm and/or the spectrum (e.g., frequency) of the sound from each microphone sensor.

Besides (or instead) of using a microphone sensor for detecting user 110's snoring, a vibrational sensor, or a nasal pressure sensor may be used for snoring detection. In an example embodiment, the vibrational sensor and/or nasal pressure sensor may be attached to user 110's nostrils to detect vibrations and/or pressure fluctuations of nostrils. Alternatively, a vibration sensor may be attached to a portion of a head, a neck, or a chest of user 110.

Various other sensors may be incorporated at a user-facing surface of patch 111 (herein, the user-facing surface is the surface configured to be directly adjacent to a skin or clothes of user 110) or at an outer-facing surface of patch 111 (herein, the outer-facing surface is the surface of patch 111 opposite to user-facing surface). For example, sensors configured to measure various other parameters associated with user 110 may be located at the user-facing surface, and sensors configured to measure various environmental parameters may be located at the outer-facing surface.

In an example embodiment, a surface airflow sensor may be used to evaluate a convective flow cooling of user 110, while a proximity sensor may detect a proximity of other surfaces (e.g., a surface of a bed, or proximity of other body surfaces) near patch 111. Additionally, patch 111 may include a photodiode for observing light condition within the room, and/or a camera for determining room orientation relative to user 110. In some cases, patch 111 may include a reflectometer for measuring reflectance of surfaces in the proximity of user 110.

As shown in FIG. 1, system 100 may include a compute device 113 configured to communicate with, and receive data from, patches 111A-111F via a communication interface. The communication interface of compute device 113 can be any suitable compute device that allows patches 111A-111F to exchange data with the compute device 113. In an example embodiment, the module may communicate with patches 111A-111F via a wireless communication (e.g., WiFi® radio, a Bluetooth® radio (e.g., a Bluetooth® antenna), a near field communication (NFC) radio, and/or a cellular radio) or a wired connection (e.g., Ethernet cable). Besides communicating with patches 111A-111F, compute device 113 may be configured to send signals to and/or receive signals from another device (e.g., a data processing device such as a cloud-based computing device, a local computing device, and the like). In some instances, the communication interface of compute device 113 can include multiple communication interfaces (e.g., a WiFi® communication interface to communicate with the one external device and a Bluetooth® communication interface to send and/or broadcast signals to another device). Further, compute device 113 may include a memory (e.g., a random-access memory (RAM)), a memory buffer, a hard drive, a read-only memory (ROM), an erasable programmable read-only memory (EPROM), and/or the like. Alternatively, or additionally, compute device 113 may include a processor for analyzing data received from sensors of patches 111A-111F.

Compute device 113 may include a memory configured to store processor executable instructions (e.g., software). As used herein, software shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions, when executed, cause a processor of compute device 113 to perform the various processes described herein. For example, the instructions stored in the memory of compute device 113 can instruct the processor to process raw data acquired from sensors of patches 111A-111F. Compute device 113 may also be configured to store data (e.g., raw data or processed data) and allow a communication interface of compute device 113 to transmit the data to another device.

Examples of compute device 113 can include a personal computer, a laptop, a tablet computer, a smartphone, a smart TV, a wearable computing device, or any other device capable of sending and receiving data.

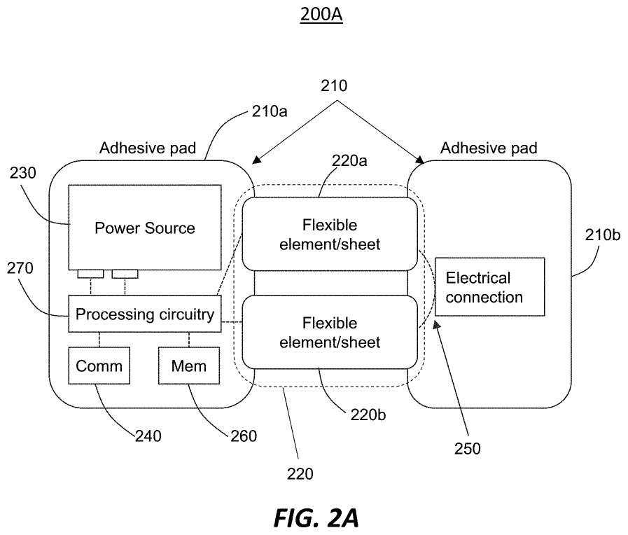

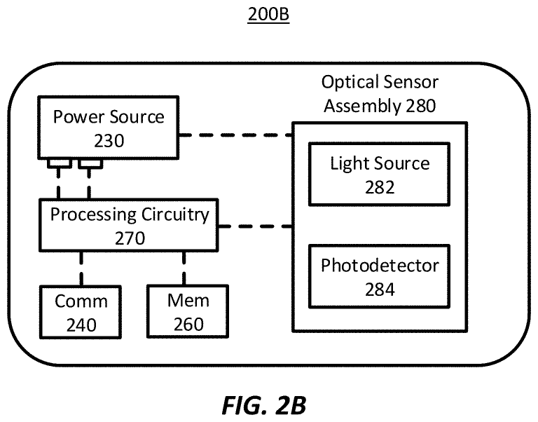

FIG. 2A shows a first example schematic illustration of an apparatus 200A (also referred to herein as a “patch”), including a processor and a communication interface for monitoring a sleep parameter of a user, in accordance with some embodiments. In an example embodiment, apparatus 200A may be an electro-mechanical and/or electro-optical part of patch 111. The apparatus 200A includes two adhesive pads 210a and 210b (collectively referred to as adhesive pad 210) connected together by a pair of flexible elements/sheets 220a and 220b (collectively referred to as element 220). In some embodiments, element 220a is a conductive element that does not exhibit piezoresistive behavior, and element 220b is an element that exhibits piezoresistive behavior. In other embodiments, both element 220a and element 220b exhibit piezoresistive behavior. Apparatuses 200A in which both element 220a and element 220b exhibit piezoresistive behavior can exhibit a greater sensing sensitivity than apparatuses 200A in which element 220a is a conductive element that does not exhibit piezoresistive behavior, and element 220b is an element that exhibits piezoresistive behavior. Element 220 can be configured to change an electrical property (e.g., resistance) in response to stress or pressure applied thereto. In addition, the two elements 220a and 220b are electrically coupled to each other via an electrical connection 250 (e.g., a wire or any other conductive link), thereby allowing electrical current to flow through the two elements 220a and 220b.

Apparatus 200A also includes a power source 230 (e.g., a battery) that is connected to a processing circuitry 270. The power source 230 is also connected to element 220 to allow the measurement of the electrical property of element 220. In some embodiments, the power source 230 can be in direct connection with element 220. In some embodiments, the power source 230 can be electrically coupled to element 220 via the processing circuitry 270.

Adhesive pad 210 can include an adhesive configured to cling firmly to the skin of a user, such that when the area of a user's skin connected to adhesive pad 210 moves, e.g., expands, contracts, rotates, and the like, relative to a starting position, a pressure or stress is applied to element 220 spanning in between the two adhesive pads 210a and 210b.

The processing circuitry 270 is connected to a communication interface 240 that is configured to communicate with another device, such as a user device. Examples of the user device can include a personal computer, a laptop, a tablet computer, a smartphone, a smart TV, a wearable computing device, or any other device capable of sending and receiving data.

The apparatus 200A also includes a memory 260 that is configured to store processor executable instructions (e.g., software). As used herein, software shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions, when executed, cause the processing circuitry 270 to perform the various processes described herein. For example, the instructions stored in the memory 260 can instruct the processing circuitry 270 to process raw data acquired from the measurement of the electrical property of the element 220. The memory 260 can also be configured to store data (e.g., raw data or processed data) and allow the communication interface 240 to transmit the data to another device.

The communication interface 240 of the apparatus 200A can be any suitable module and/or device that can place the resource in communication with the apparatus 200A such as one or more network interface cards or the like. Such a network interface card can include, for example, an Ethernet port, a WiFi® radio, a Bluetooth® radio (e.g., a Bluetooth® antenna), a near field communication (NFC) radio, and/or a cellular radio. As such, the communication interface can send signals to and/or receive signals from another device. In some instances, the communication interface of the apparatus 200A can include multiple communication interfaces (e.g., a WiFi® communication interface to communicate with the one external device and a Bluetooth® communication interface to send and/or broadcast signals to another device). The memory 260 can be a random-access memory (RAM), a memory buffer, a hard drive, a read-only memory (ROM), an erasable programmable read-only memory (EPROM), and/or the like.

The processing circuitry 270 can include any suitable processing device configured to run or execute a set of instructions or code (e.g., stored in the memory) such as a general-purpose processor (GPP), a central processing unit (CPU), an accelerated processing unit (APU), a graphics processor unit (GPU), an Application Specific Integrated Circuit (ASIC), and/or the like. Such processing circuitry 270 can run or execute a set of instructions or code stored in a memory associated with using a PC application, a mobile application, an internet web browser, a cellular and/or wireless communication (via a network), and/or the like.

The processing circuitry 270 can be realized as one or more hardware logic components and circuits. For example, and without limitation, illustrative types of hardware logic components that can be used include general-purpose microprocessors, microcontrollers, digital signal processors (DSPs), and the like, or any other hardware logic components that can perform calculations or other manipulations of information.

In operation, the apparatus 200A can be configured to measure the respiratory effort exerted by a user via the piezoresistive effect. The respiratory effort can be represented, for example, as a voltage (e.g., μV, mV, or V). A voltage is applied by the power source 230 across the element 220, and a certain resistance (e.g., initial resistance) is introduced. When the user's skin is expanded or contracted, the element 220 reacts by expanding or contracting, respectively, thereby inducing changes in the electrical property. Such changes are captured by the processing circuitry 270 and associated with a user movement, such as how much a user's chest is rising and falling.

FIG. 2B shows a second example apparatus, 200B, for obtaining sleep data during a sleep of a user, according to some embodiments. As shown in FIG. 2B, the apparatus 200B includes a power source 230 configured to supply power to processing circuitry 270, a communication interface 240, a memory 260, and an optical sensor assembly 280, which includes one or more light sources (collectively, light source 282) and a photodetector 284 (e.g., a photovoltaic cell). In some embodiments, the light source 282 is configured to emit red or infrared light. Alternatively or in addition, the light source 282 can be configured to emit light at any other wavelength. The light source 282 can be controllable by a controller and/or other electronics onboard, or in wired or wireless communication with the onboard electronics. The optical sensor assembly 280 can be incorporated into any patch/apparatus of the present disclosure. The apparatus 200B is formed as a single patch, which can be applied (e.g., adhered, via an adhesive surface thereof) to a surface of a wearer for use. During use, at least a portion of the light emitted from the light source 282 reflects off the skin of the wearer and is detected by the photodetector 284.

In some embodiments, systems, devices, and methods disclosed herein may comprise one or more systems, devices, and methods such as those described in the U.S. Pat. No. 10,531,832B2, filed on Oct. 5, 2018, and titled “SYSTEMS, APPARATUS, AND METHODS FOR DETECTION AND MONITORING OF CHRONIC SLEEP DISORDERS,” the contents of which are hereby incorporated by reference in their entirety.

The movements can be correlated to the respiratory effort or the breathing rate of a user. Analyzing the respiratory effort can reveal information about the breathing and/or sleep issues of the user. For example, it may be determined that the normal respiratory rate is about 12-16 per minute for an adult, 15-25 per minute for a child, and 20-40 per minute for an infant. Rates above or below these ranges may be determined as indication of abnormal conditions of the user. In another example, the movements can be correlated to the respiratory effort of the user, indicating possible difficulty in breathing as a result of partial or full blockage of one of the user's air paths. The respiratory effort measurement is also a useful parameter in detecting one of the most common and severe sleep disorders, sleep apnea.

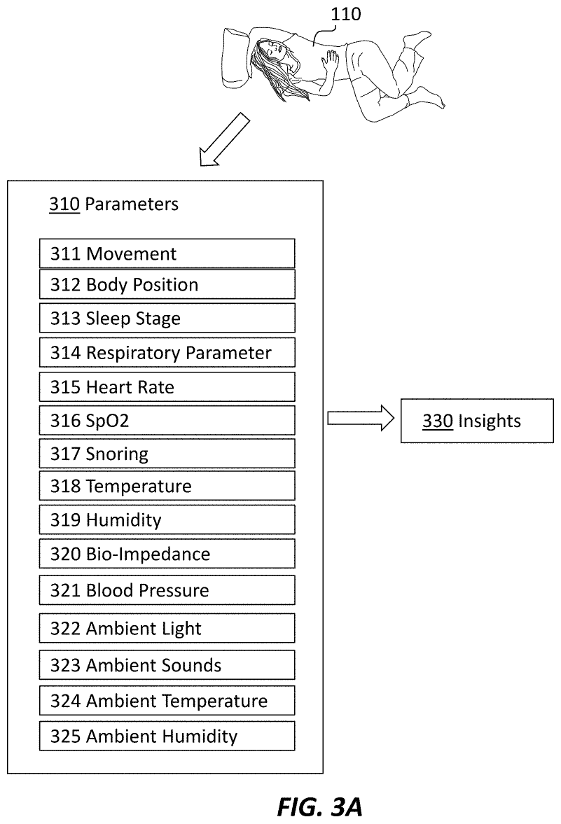

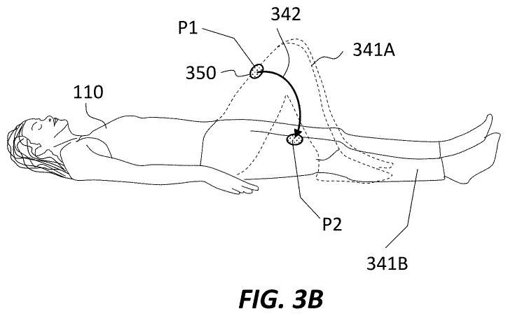

FIG. 3A shows various parameters 310 that may be collected from sensors of patches attached to user 110. For example, a movement 311 parameter describing motions of a body of user 110 may be collected. The motions of user 110's body may include any suitable motions such as movement of limbs (arms or legs), movement of a head of user 110, or any movements of a torso (such as rotations, bending or twisting of the torso). In some cases, movements may include body seizures, or vibrations, such as tremors. Movement 311 may be represented as a coordinate transformation of selected points on a body. In an example embodiment, various points on a body may be selected, and coordinates of these points may be tracked via measurements obtained from one or more patches (e.g., patches 111A-111F) containing accelerometers. Based on the motion of these selected points a change in body's position may be reconstructed. For example, a movement of a selected point located on a head of user 110 can be used to track rotations of the head of user 110 and/or bending of user 110's neck. Additionally, relative movements of various selected points may be used to determine overall changes in a position of a body of user 110.

FIG. 3B shows an example movement of point 350 located at a position P1 (point 350 may be coincident with a patch attached at position P1) to a position P2 as indicated by an arrow 342. The motion of point 350 may occur when user 110 moves her legs from a bent position 341A to an extended position 341B. It should be appreciated that various other points may be tracked to establish the body motions of user 110. For example, system 110 may be configured to track movements of a torso of user 110, movements of limbs of user 110, as well as movements of a head and a neck of user 110. In some cases, movements of hands and feet may be tracked as well, including movements of fingers and toes. In some cases, movements of the chest may be tracked due to breathing. Further, movements of facial features may be tracked as well (e.g., movements of eyes may be tracked via electrodes attached to a facial area in the proximity of user 110's eyes), or via any other suitable approaches.

Compute device 113 may further collect body position 312 data collected from various sensors of patches 111A-111F. In some cases, determining a position of a body of user 110 may be obtained without tracking the body motions of user 110. For instance, whether user 110 is in an upright or horizontal position may be obtained directly from accelerometers of one or more patches 111A-111F without determining motions of user 110's body.

Additionally, based on a combination of parameters, a sleep stage 313 may be determined by compute device 113 (or any other device associated with compute device 113, such as, for example, a cloud-based computing device). The parameters can include (but are not limited to), for example, one or more of: a motion of the eyes of user 110, a frequency of body movements of user 110, pulse measurements for user 110, audio measurements of microphone sensors, one or more breathing patterns of user 110, one or more breathing disturbances of user 110, a breathing quality of user 110, and/or the like. The sleep stage 313 can include, for example, a light sleep stage, a deep sleep stage, a rapid eye movement (REM) stage, a non-rapid eye movement (NREM) sleep stage, or a wake stage. A REM sleep stage can include tonic and phasic components. The tonic component can be characterized by relatively slow changes in a galvanic skin response (GSR) signal, with the change occurring, for example, on a scale of tens of seconds to minutes. The phasic component, on the other hand, can be characterized by relatively rapid changes in the GSR signal (e.g., on the order of seconds). Such rapid changes are known as skin conductance responses (SCRs) and manifest themselves as rapid fluctuations or peaks that can be observed in a GSR signal. It should be noted that tonic and phasic components may be part of the same REM sleep stage. A NREM sleep stage can include a light sleep stage (e.g., NREM N1 or NREM N2) or a deep sleep/slow-wave sleep stage (e.g., NREM N3). Any of the sleep stages and sleep stage components described herein may be determined by compute device 113 and/or by any other device associated with compute device 113, such as, for example, a cloud-based computing device).

Further, besides determining sleep stage 313, the combination of parameters determined by compute device 113 (or any other device associated with compute device 113) may be used for determining a breathing pattern of a user, which may be characterized by a rate of a breathing of the user, by a depth of the breathing of the user, and/or by a frequency of breathing disturbances and/or types of breathing disturbances. The types of breathing disturbances may be classified as apnea, hypopnea, eupnea, orthopnea, dyspnea hyperpnea, upper airway resistance, hyperventilation, hypoventilation, tachypnea, Kussmaul respiration, Cheyne-Stokes respiration, sighing respiration, Biot respiration, apneustic breathing, central neurogenic hyperventilation, central neurogenic hypoventilation, or any other type of breathing disturbances known in the art. The frequency of breathing disturbances may range from a breathing disturbance occurring every few seconds to a breathing disturbance occurring every few minutes, every few tens of minutes, or every one or more hours of sleep including all the values and ranges in between a few seconds to a few hours. In some cases, the breathing disturbance may occur for every breath of a user, or may happen according to a regular pattern (e.g., for every few breaths of the user), or may happen irregularly. In some cases, the breathing disturbance may occur for every inhalation of the user or for every few inhalations of the user. Additionally, or alternatively, the breathing disturbance may occur for every exhalation of the user, or for every few exhalations of the user.

Additionally, besides determining sleep stage 313, the combination of parameters determined by compute device 113 (or any other device associated with compute device 113) may be used for determining whether a user is awake. Further, the breathing pattern of a user may be determined when the user is asleep or awake.

Additionally, or alternatively, the combination of parameters may be used to determine if the user is in a hypnagogic or hypnopompic stage, and/or experiencing hypnagogic hallucinations, lucid thought, lucid dreaming, and/or sleep paralysis. In some cases, the combination of parameters may indicate that the user is in unconscious or under anesthesia.

In an example embodiment, compute device 113 may be configured to collect, detect, or determine one or more respiratory parameters 314 such as, for example, an overall respiratory effort, a breathing depth, a frequency of breathing, a respiratory flow, and/or a respiratory pressure. For example, the one or more respiratory parameters 314 may be determined by sensors associated with patch 111 placed adjacent to a chest of a user. Further, the one or more respiratory parameters 314 may include breathing sound parameters collected by one or more microphones associated with patch 111. In some cases, microphones may detect wheezing or any other sounds emanating from a chest area of user 110. In an example implementation, one or more microphones may be associated with device 113, or with any other suitable external device. Further, user 110's nasal airflow and/or air nasal pressure sensors may be used to further parameterize respiratory effort as part of the one or more respiratory parameters 314. Such measurements may determine that user 110 suffers from an apnea or a hypopnea (e.g., by monitoring changes to sensed signals related to nasal airflow and/or air nasal pressure).

When collecting, detecting, or determining one or more respiratory parameters 314, compute device 113 may determine a respiratory quality (herein, respiratory quality refers to a degree of relaxation during breathing). To assess the respiratory quality, a use of accessory muscles in the neck and chest and indrawing of intercostal spaces and movement of intercostal muscles may be analyzed by suitable sensors of patches 111 (e.g., vibration and stiffening of user 110's body may be analyzed via piezoelectric sensors) to determine a level of relaxation during breathing of user 110. Further, compute device 113 may determine a respiratory rate (e.g., how many breaths are taken per minute) and a regularity of the respiratory rhythm of user 110. In some cases, when respiratory rate is outside the expected respiratory rate for user 110 (the expected respiratory rate for user 110 may be calibrated based on an age and size of user 110), an alarm can be generated by an alarm device of system 100 to alert user 110. Additionally, or alternatively, when a respiratory rhythm is outside the expected respiratory rhythm for user 110 (the expected respiratory rhythm for user 110 may be calibrated based on an age and size of user 110), an associated alarm can also be generated by an alarm device of system 100 to alert user 110. Further, when collecting, detecting, or determining one or more respiratory parameters 314, a sum-flow (e.g., a measure of air flow derived from two measures of respiratory effort (one from the abdomen, one from the thorax)) may be determined. In an example embodiment, a sum-flow is computed as a gradient of a sum of respiratory effort signals. Sum-flow may be used to assess one or more sleep characteristics of user 110 (e.g., to determine whether user 110 has a sleep apnea).

In various embodiments, data (e.g., respiratory parameters 314 or any other parameters related to a user's sleep) collected for each night of sleep may be further aggregated to present sleep trends over time. For example, for the parameters (e.g., data) that are being collected, trends may be determined and presented to a user and/or to a medical professional in the form of tables, graphs, histograms, or any other suitable manner. Parameters collected can include, but are not limited to, one of or any combination of: respiratory parameters 314, parameters indicating an overall sleep quality for each night, parameters indicating a sleep quality for a given monitored period, parameters indicating an overall sleep time/duration for each night, parameters indicating a sleep time/duration for a given monitored period, parameters indicating an overall sleep efficiency for each night, parameters indicating a sleep efficiency for a given monitored period, parameters indicating a sleep position or sequence of sleep positions for each night, parameters indicating a sleep position or sequence of sleep positions for a given monitored period, parameters indicating a frequency of “wakes” or sleep disruptions for each night, parameters indicating a frequency of “wakes” or sleep disruptions for a given monitored period, parameters indicating a frequency of respiratory disturbances (e.g., associated with or indicative of apnea hypopnea index (AHI), respiratory disturbance index (RDI), and/or respiratory event index (REI)) for each night, parameters indicating a frequency of respiratory disturbances (e.g., associated with or indicative of AHI, RDI, and/or REI) for a given monitored period, parameters indicating a frequency of oxygen desaturation (e.g., associated with or indicative of an oxygen saturation index (ODI)) for each night, parameters indicating a frequency of oxygen desaturation (e.g., associated with or indicative of ODI) for a given monitored period, parameters indicating an oxygen saturation profile (e.g., mean, medium, minimum oxygen saturation (“SpO₂”), maximum SpO₂, and/or T90 (i.e., sleep time spent with an SpO₂ of <90%)) during sleep for each night or for a given monitored period, parameters indicating an overall breathing pattern for each night, parameters indicating a breathing pattern for a given monitored period, parameters indicating an overall rate of occurrence of snoring for each night, parameters indicating a rate of occurrence of snoring for a given monitored period, parameters indicating cardiac cycles, and GSR related parameters. In some cases, the trends may be established after a suitable data analysis. The suitable data analysis may include data extrapolation, data interpolation, pattern recognition, data analysis using machine learning approaches (e.g., using suitable neural networks for classifying and analyzing data, and/or the like). The data may be analyzed separately for each one of the nights for which the data is collected, or can be analyzed as an aggregated data (e.g., analyzed for all of the nights for which the data is collected). In some cases, the data may be analyzed for groups of nights (e.g., a first group of nights may be nights of Friday and Saturday, while the second group of nights may be nights between and including Sunday and Thursday).

Further, the impact of various interventions and changes in user behavior/therapy may be analyzed to determine an effect thereof on user's sleeping trends. For example, a statistical correlation between the changes in sleeping trends of the user and changes in user behavior may be analyzed to determine beneficial behavioral changes (e.g., not using electronic devices before sleeping, reducing food consumption before sleeping, exercising a few hours before sleeping, and the like) and detrimental behavioral changes (e.g., consuming caffeine before sleeping). Alternatively or in addition to statistical or other numerical analyses, a user and/or physician can determine anecdotally, via observation, whether certain interventions and/or changes in user behavior/therapy have impacted the user's sleep.

As described above, at least some of the sensors associated with patch 111 may collect heart rate 315 parameters and/or oximetry data (referred to herein as SpO₂ 316). Further, as described above, the sensors may also be configured to collect audio/vibrational data due to snoring (herein, referred to as snoring 317), body temperature data (herein, referred to as temperature 318), body humidity data (herein, referred to as humidity 319), or bio-impedance 320 parameters (e.g., bio-impedance may be used to determine a humidity of skin of user 110)

In various embodiments, compute device 113 or any other suitable compute device may be configured to emit audio and/or visible signals. For example, compute device 113 may emit calming sounds, calming light patterns, and the like. In an example embodiment, a relationship between calming sounds/lights and user sleep characteristics may be detected within, and stored by, system 100. In an example embodiment, compute device 113 may collect data related to ambient light 322 and/or ambient sounds 323, and detect or calculate a relationship between the ambient light and/or ambient sounds and the user sleep characteristics. Further, compute device 113 may be configured to control an ambient temperature 324 and/or ambient humidity 325, for example by generating and transmitting a control signal to a heating, ventilation and air conditioning (HVAC) controller, a thermostat, a humidifier, a temperature controller, etc., to cause a change in temperature and/or humidity thereof.

In some implementations, system 100 includes an additional device or component for measuring a blood pressure 321 of user 110. For example, the additional device may be a sphygmomanometer that may include an inflatable cuff. In some cases, patch 111 may be equipped with blood pressure measuring sensors (e.g., such sensors may be ultrasound transducers configured to measure changes in blood vessels' diameters due to changes in blood pressure).

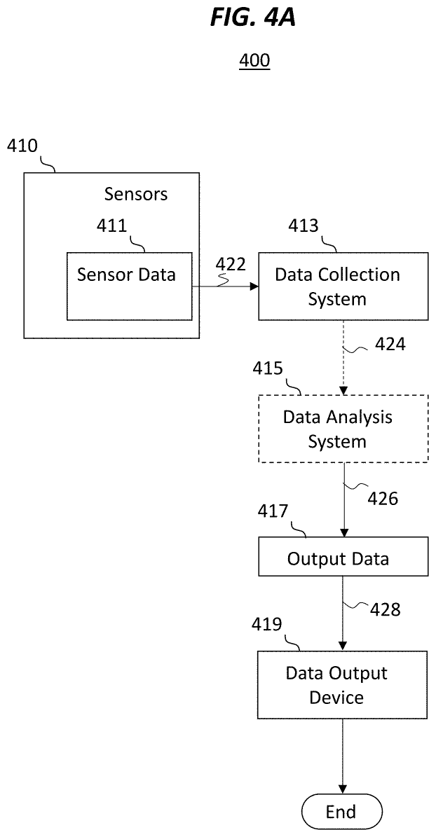

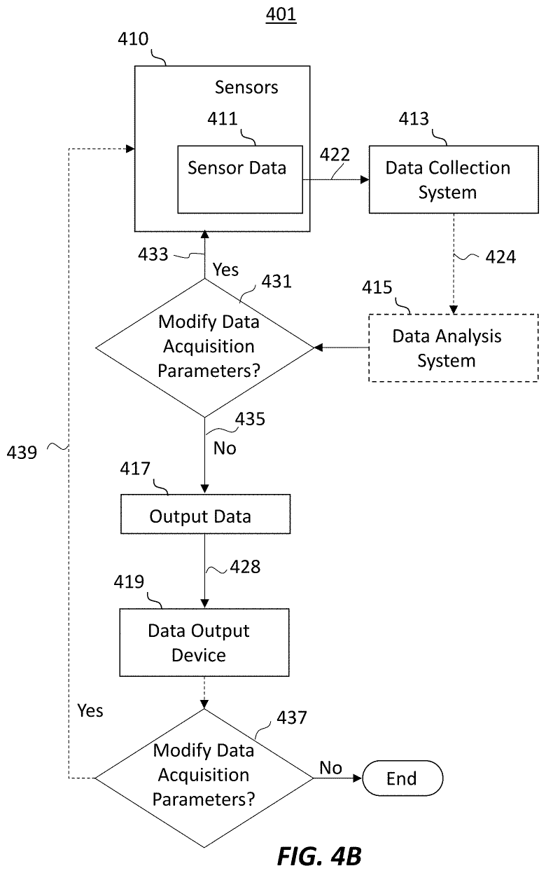

System 100 may be configured to process parameters 310 and provide insights 330, which may include an animation of user positions, a list of favorable positions, times when user snored, and the like, as further discussed below. FIG. 4A shows an example diagram 400 for collecting and processing sensor data. In an example embodiment, sensors 410 (sensors 410 are associated with patches 111A-111F) are configured to collect sensor data 411 (sensor data includes one or more parameters 310) and transmit data 411, at step 422, to a data collection system 413 (e.g., compute device 113, as shown in FIG. 1). Data collection system 413 may be configured to process collected sensor data 411 (e.g., combine data, compress data, discard erroneous data, and the like). In an example embodiment, system 413 may transmit processed data at step 424 to a designated data analysis system 415. Data analysis system 415 may be a cloud-based computing system, a local computer, or any other suitable computing resource for processing data. Data analysis system 415 includes one or more processors configured to analyze received data. Further, data analysis system 415 may include any suitable memory devices for storing software instructions, as well as various received data (or any other data). Such analysis includes generating images of positions of a body of user 110. Further, one or more processors of system 415 may be configured to perform statistical analysis of sensor data 411, and/or generate various time plots associated with sensor data 411. In some cases, one or more processors of system 415 may be configured to detect changes in sensor data 411 and identify key events during a sleep of user 110, as further described below. In some cases, data collection system 413 may include a processor configured to perform various data analysis operations, such a generating images of positions of a body of a user, or any other operations that may be, otherwise, performed by data analysis system 415. Alternatively, data analysis system 415 may be part of data collection system 413. At step 426, data analysis system 415 may generate output data 417 (output data 417 includes results of the data analysis, such as an animation of positions of the body of user 110, data statistics, time plots, and the like), and at step 428 transmit output data 417 to a suitable output device 419. In an example embodiment, output device 419 may be any suitable device for presenting data for a user. A non-exhaustive list of output devices may include a display (e.g., a touch screen of a smartphone, a computer monitor, a projector image, and virtual reality headset, and the like), and audio device (e.g., a speaker, a smart speaker, such as Alexa, a headset, and the like), a paper copy, and the like. In one embodiment, output device 419 may be a device associated with user 110 (e.g., a smartphone). Additionally, or alternatively, output device 419 may be associated with a physician, or any suitable third party (e.g., a medical insurance provider, a hospital, a home-care provider, a nurse, a medical equipment provider, and the like) that is authorized to access output data 417. In an example embodiment, output device 419 may be part of compute device 113. In some cases, data analysis system 415 (or data collection system 413) may be configured to transmit output data 417 to a plurality of output devices. In some cases, output data 417 may be stored on a server (e.g., a cloud-based server) and may be accessible by one or more electronic devices configured to display output data 417. In some cases, a suitable application programming interface (API) may be used for accessing and displaying output data 417.

FIG. 4B shows diagram 401, which is a variation of diagram 400. Diagram 401 includes elements 410, 411, 413, 415, 417, and 419, which are the same as the same numbered elements of diagram 400. Also, steps 422, 424, and 428 are the same as the same numbered steps of diagram 400. Additionally, after processing data, data analysis system 415 may be configured to determine at step 431 if one or more data acquisition parameters need to be modified. Modifying data acquisition parameters may include changing a frequency at which sensors 410 acquire various parameters 310, determining which one of sensors 410 needs to acquire data, determining one or more time delays between multiple sensors from sensors 410 for acquiring data, determining logical rules for acquiring data, and the like. An example logical rule may include acquiring a pulse data from a first sensor if a breathing frequency is higher than a threshold target value. Any other logical rules that relate acquisition of data of one sensor based on data obtained from another sensor may be used. Such logical rules may be determined by data analysis system 415 based on data output requirements. For example, if data output requirements include displaying the pulse rate if the breathing frequency is higher than the threshold target value, a corresponding logical rule described above may be used.

If one or more data acquisition parameters need to be modified (step 431, Yes), acquisition parameters may be modified at step 433 and new sensor data 410 may be collected. Alternatively, if no changes in data acquisition are needed (step 431, No), output data 417 may be output at step 435. Further, at step 437, after displaying data, user 110 or a medical professional (e.g., physician, nurse, etc.), may determine that changes in data acquisition are needed. If such changes are needed (step 437, Yes), acquisition parameters may be modified at step 439. Alternatively, if no changes in data acquisition are needed (step 437, No), no changes in acquisition parameters are made.

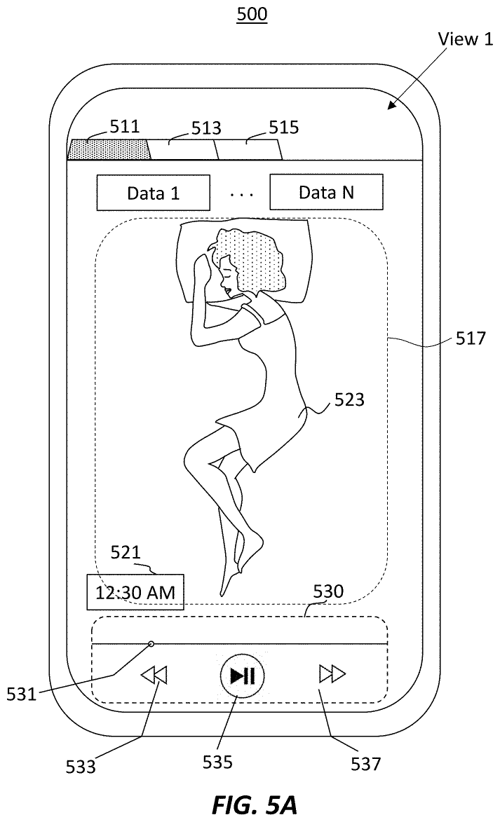

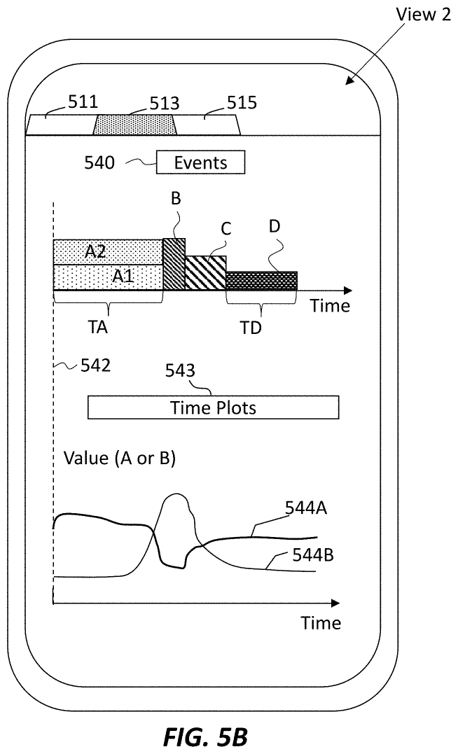

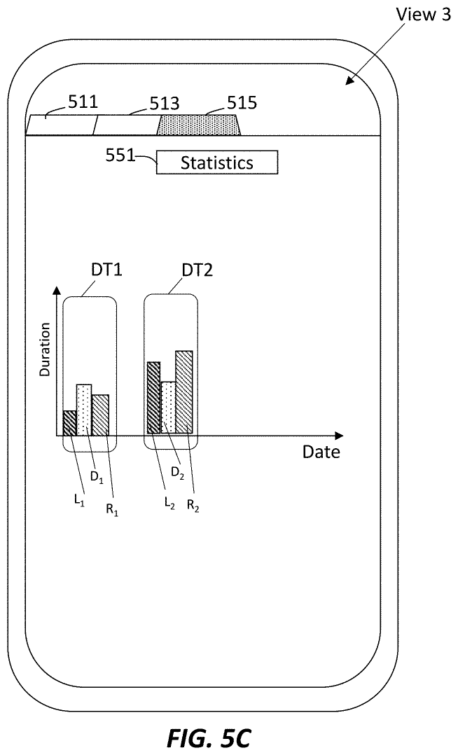

FIG. 5A shows an example interface 500 of data output device 419. In an example embodiment, interface 500 may include graphical user interface (GUI) elements, such as tabs 511, 513, and 515, data displaying elements Data 1 through Data N, a region 517 for displaying images or animated motions (herein, referred to as animation or body animation) of a body of user 110, a time element 521 for displaying time at which the body position and Data 1 through Data N are recorded, as well as animation controlling elements 530. Animation controlling GUI elements may be typical GUI elements for controlling video data, such as a time scroll 531, fast forward element 537 for moving the animation forward, fast backwards element 533 for moving the animation backward, and play/pause toggle element 535. Any other suitable GUI elements for controlling animation may be used as well. In an example embodiment, the animation is shown in region 517 by depicting body positions of user 110 as a function of time, as indicated by GUI element 521. In various embodiments, data displaying elements Data 1 through Data N may be configured to display any suitable parameters 310, as recorded by sensors 410. For example, Data 1 may show blood oxygen levels, Data 2 may show whether user 110 was/was not snoring, and another data displaying element (e.g., Data 3) may show a pulse rate of user 110. Any other parameters characterizing a sleep of user 110 may be displayed as well via data displaying elements Data 1 through Data N.

In some cases, interface 500 may be a touch screen allowing a user to interact with GUI elements of interface 500. Additionally, or alternatively, a user may interact with interface 500 via any other suitable means (e.g., via a mouse, a keyboard, audible sounds, user gestures, and the like). In an example embodiment, a user may toggle between different tabs 511-515 to select different views (e.g., View 1 through View 3, as shown in corresponding FIGS. 5A and 5C) of output data. For example, FIG. 5A shows GUI elements associated with tab 511, FIG. 5B shows GUI elements associated with tab 513, and FIG. 5C shows GUI elements associated with tab 515. It should be noted that a usage of tabs 511-515 is only one possible illustrative way of selecting different views, and any other suitable GUI elements (e.g., lists, buttons, etc.) may be used. Additionally, or alternatively, user commands (e.g., text commands entered into a command prompt, audible sounds, gestures, and the like) may be used to switch between different views.

FIG. 5B shows an example view (View 2, which may be associated with tab 513) depicting events 540, such as events A1, A2, and B-D associated with a sleep of user 110. In an example embodiment, events are depicted as a function of time duration and may be characterized by bars of different colors (or patterns) and/or different amplitude (when a notion of an amplitude is applicable for the event). For example, event A1 has a duration of TA and may be associated with user 110 sleeping on her/his back, while event D has a duration of TD, and may be associated with an increased pulse rate of user 110. For such an example, event D has an associated amplitude (e.g., a rate of pulse) which may be obtained by clicking on a GUI element associated with event D. In an example embodiment, event A2 may correspond to a decreased blood oxygen levels and may occur at the same time as (or overlap in time with) event A1.

View 2 may include time plots 543 of various parameters 310. In an example embodiment, the time axis for time plots 543 and events 540 may be aligned as indicated by dashed line 542. As shown in FIG. 5B, time plots 543 may include more than one time plot (e.g., time plot 544A and 544B). For example, time plot 544A may indicate a pulse rate, and time plot 544B may indicate an amplitude of a sound associated with user 110 snoring.

FIG. 5C shows another example view (View 3, which may be associated with tab 515) for displaying statistics 551 associated with parameters 310 for different dates DT1 and DT2. For example, for date DT1, the L1 element may be associated with a light sleep stage, D1 element may be associated with a deep sleep stage and R1 element may be associated with a REM sleep stage. Alternatively, for date DT1, the L1 element may be associated with a wake stage, D1 element may be associated with a light sleep stage and R1 element may be associated with a deep sleep stage. In an example embodiment, a height of elements L1, D1, and R1 may indicate the duration of time of the sleep stage. Similarly, elements L2, D2, and R2 may correspond to light, deep and REM sleep stages for date DT2. Although shown and described herein (e.g., in FIG. 5C) as including three sleep stages per monitored time period, any other suitable number of sleep stages (e.g., one, two, four, five, six, seven, eight, nine, ten, etc.) can be monitored and can have associated statistics generated and displayed via the GUI interface in a histogram, pie chart, bar chart, linear plot, and/or any other suitable format.

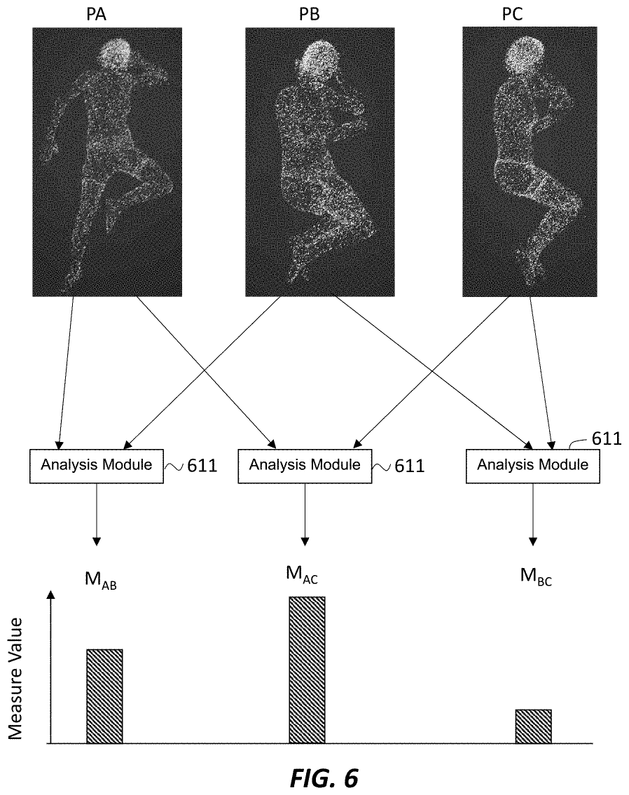

FIG. 6 shows illustrative body positions PA, PB, and PC of user 110 at different times during a sleep of user 110. For example, PA is a position of user 110 laying substantially facing down, PB shows user 110 laying partially on her/his side and partially on her/his back, while PC shows user 110 laying on her/his side. In an example embodiment, data analysis system 415 (as shown in FIGS. 4A and 4B) may include an analysis module 611 for comparing a pair of positions. In an example embodiment, analysis module 611 may compare positions PA and PB and generate a numerical score M_{AB} (herein, also referred to as a score, a measure function, a measure value, or a measure score) qualifying a difference between the positions PA and PB. Similarly, when comparing positions PA and PC, a measure score M_{AC} is generated and when comparing positions PB and PC, a measure score MBC is generated. In an example embodiment, a value of, for example, M_{AC} indicates how different are positions PA and PC. As shown in FIG. 6, measure score M_{AC} may have a larger value than M_{AB}, indicating that positions PA and PC are more different from each other than positions PA and PB. Similarly, positions PB and PC may be similar resulting in a low value of MBC, as shown in FIG. 6.

Analysis module 611 may receive various sensor data from sensors 410 (as shown in FIGS. 4A and 4B) and may calculate a measure score in any suitable way. In an example embodiment, analysis module 611 may estimate coordinates of various points on a surface of a body of user 110. For example, for position PA, a first set of coordinate vectors {r_{Ai}} may be used and a second set of coordinate vectors {r_{Bi}} may be used for position PB. Using these sets of coordinates, an example measure score M may be calculated using an amplitudes of differences between {r_{Ai}} and {r_{Bi}}. For example, M=Σᵢ(r_{Ai}−r_{Bi})·(r_{Ai}−r_{Bi}). It should be noted that any other suitable approach may be used for calculating measure score M. For example, analysis module 611 may be a machine-learning model (e.g., any suitable neural network model) configured to determine differences in body positions of user 110 based on sensor input data. In some cases, the machine-learning model may be tailored based on user 110 characteristics such as a height of user 110, weight of user 110, or any other suitable personal characteristics (e.g., a size of a head of user 110). In some embodiments, measure score M may be a single number, but in other cases, measure score M may be a list of numbers. For example, measure score M may include a list of numbers, M={m_{H}, m_{LA}, m_{RA}, mₐ, m_{RL}, m_{T}, m_{S}} for determining fine differences between positions of a body of user 110, with m_{H} indicating a measure score for a difference in a position of a head, m_{LA} indicating a measure score for a difference in a position of a left arm, m_{RA} indicating a measure score for a difference in a position of a right arm, m_{LL} indicating a measure score for a difference in a position of a left leg, m_{RL} indicating a measure score for a difference in a position of a right leg, m_{T} indicating a measure score for a difference in a position of a torso, and m_{S} indicating a measure score for a difference in a position of shoulders of user 110.

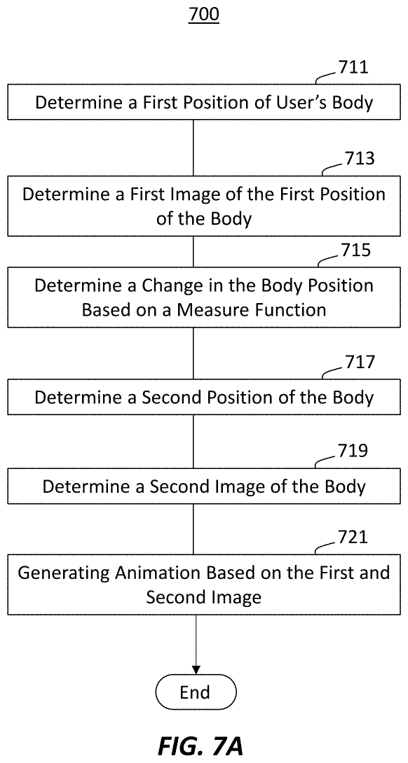

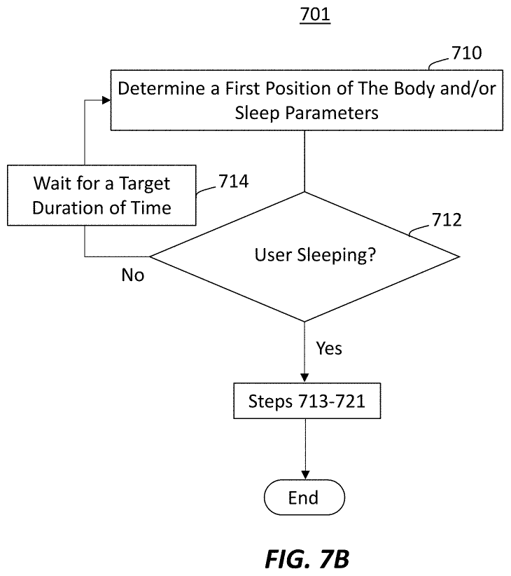

FIG. 7 shows an example process 700 for generating an animation, consistent with disclosed embodiments. Steps 711-721 of process 700 may be performed by data analysis system 415. At step 711 of process 700, system 415 may determine a first position of a body of user 110 based on data from sensors 410. Determining the first position may include recording the first position in a memory device associated with system 415. At step 713, based on the first position of a body, an associated first image of the position of the body is determined. The first image may be stored in the memory device associated with data analysis system 415. At step 715, system 415 continuously (or periodically) analyzes positions of the body of user 110 by analyzing data continuously (or periodically) received from sensors 410. Further, at step 715, system 415 continuously (or periodically) evaluates measure score M to detect a change in a position of the body of user 110. In an example embodiment, measure score M may be calculated to detect a difference between positions of a body as a function of time (i.e., body positions at a first and a second time are determined and a difference in these positions is evaluated via measure score M). At step 717, If measure score M is above a target threshold value (the target threshold value may be selected by a data analysis system 415, a medical practitioner, or user 110), data analysis system 415 may determine that user 110 is moved to a second position. The second position, then may be recorded (herein, also referred to as determined) in the memory associated with system 415, and, at step 719, based on the second position of a body, an associated second image of the position of the body is determined. The second image may be stored in the memory device associated with data analysis system 415. At step 721, the first and second images may be used for the generation of an animation, which may be displayed via interface 500, as described above. It should be noted that process 700 may be continuously performed during a sleep of user 110, resulting in collecting multiple body positions, with associated images used for generating the animation. In an example embodiment, the animation includes a representation of at least one of pressure data, breathing data, pulse data, blood oxygen level data, temperature data, or a snoring condition of the user. Further, system 415 may be configured to estimate the sleep stage of the user based on a frequency of change in a position of the body of the user. The sleep stage of the user may also be presented as a part of the animation. In various embodiments, system 415 may be configured to detect a significant change in sensed data, the sensed data including one of the pressure data, breathing data, pulse data, blood oxygen level data, temperature data, or snoring data, based on a predefined threshold.

FIG. 7B shows a process 701, which may be a variation of process 700. In an example embodiment, at step 710 of process 701, data analysis system 415 may be configured to determine the first position of a body of user 110 based on data from sensors 410. Further, at step 710, system 415 may be configured to collect various other sleep parameters 310 (previously shown in FIG. 3A) for determining various characteristics of user's sleep (e.g., sleep parameters 310 in addition to (or instead of) data associated with the first position of the body may allow for determination of whether user 110 is sleeping). At step 712 of process 701, system 415 may be configured to determine if user 110 is sleeping. For instance, data analysis system 415 may determine that user 110 is sleeping based on the determination as to whether the user is in a vertical position, a seated position, or a horizontal position, and/or at least one of breathing data or pulse data. In an example embodiment, pulse data and a position of a body of user 110 may be determined at a selected first time interval. In some cases, one or more processors of system 415 may be also configured to determine whether user 110 is sleeping further based on a change in breathing data or a change in pulse data during a second time duration subsequent to the first time duration. System 415 may be configured not to process positional data when the processor determines that the user is not sleeping. For example, if it is determined that user 110 is sleeping (step 712, Yes), system 415 may proceed to steps 713-721 of process 700. Alternatively, if it is determined that user 110 is not sleeping (step 712, No), system 415 may, at step 714, wait for a target duration of time, and then proceed to step 710. In case when a determination of whether user 110 is sleeping or not is inconclusive, system 415 may proceed to steps 713-721 of process 700.

In some cases, data analysis system 415 may be configured to determine actigraphy parameters based on data collected from sensors 410 (or from other sensors). In an example embodiment, to collect actigraphy parameters, system 100 may include a wrist-based device attached to a wrist of user 110. In an example embodiment, the wrist-based device may include patch 111, or may be any other suitable device (e.g., a wristwatch, an Apple watch, and the like). In an example embodiment, patch 111 may be configured to be placed over a wrist of user 110 and may partially wrap the wrist of user 110. Actigraphy parameters may include overall activity of user 110 (e.g., whether user 110 is in upright position, whether user 110 is walking, and the like). In some cases, actigraphy parameters include determining how often user 110 is moving her/his arms. In various embodiments, actigraphy data may be used with or without other sleep-related parameters, such as a heart rate and respiratory effort data, to assess sleeping patterns for user 110.

As described herein, since the generated animation is configured, in some embodiments, to show changes in a position of a body of a user, the animation can be a time lapse animation. In various embodiments, accelerometer data recorded from different patches is transmitted to an application run on a compute device 113 (e.g., a mobile software application (“app”) run on a smartphone) and, subsequently, may be uploaded to a server. In an example embodiment, the data is recorded at a sampling frequency of a few cycles per second or Hertz (Hz). For example, the data may be recorded at about 1 Hz, about 5 Hz, about 10 Hz, about 15 Hz, about 20 Hz, and the like. In some cases, the data may be collected with a frequency of between about 1 Hz and about 100 Hz. Alternatively or in addition, the data may be collected with a desired or predefined “resolution” (defined as the number of bits used when measuring and storing the data). For example, the data may be collected with a sampling frequency of at least about 10 Hz and a resolution of at least 16 bits, or the data may be collected with a sampling frequency of at least about 100 Hz and a resolution of at least 18 bits.

In some cases, a user (e.g., user 110) may start and stop a session for collecting sleep data. For example, user 110 may first attach patches 111A-111F and then start the session vi an application run on compute device 113. In an example embodiment, the application may be configured to communicate with electronic components of patches 111A-111F to activate sensors of patches 111A-111F for collecting data. In some cases, as described, for example, by process 701, system 100 may be configured to collect data when user 110 is sleeping, and may not collect data when user 110 is not sleeping (e.g., when user 110 is preparing for the night, is walking, talking, leaning in an armchair, eating, waking up in the middle of the night, and the like). In some cases, system 100 may be configured to allow user 110 to set up a start timer at which the data collection starts. For example, if user 110 is expecting to fall asleep at about 11:00 pm, user 110 may set a timer at that time. In some cases, system 100 may be configured to allow user 110 to set up a stop timer at which the data collection stops. For example, user 110 may set up the stop timer in the morning.

In various embodiments, as described above, a generated animation shows at least some (or each) possible position transition (e.g., from user 110 laying on a right side to user laying on a left side, or from left side to supine etc.). The generated animation (herein also referred to the generated video) may include a pre-rendered video (herein, also referred to as a prefix video). The prefix video may be a few second video showing a black background with information related to some sleep parameters of user 110. In some cases, prefix video may show an introductory text, image, sound, graphical user interface, or combination thereof (e.g., the text may be “Here is a quick summary of your night” or any other similar introductory text).

In an example embodiment, a session transitions table is generated to summarize all of the transitions associated with user 110 changing a position of user 110's body during a sleep session. In an example embodiment, session transitions table is generated by dividing the session into predefined number of time intervals (herein, also referred to as time windows) and finding a position of user 110's body for each time window. By way of example, the process of dividing the session into the time intervals and finding the position of user 110's body may be implemented using the following pseudo-code:

```
timeChunks = divideTimeIntoChunks
(time, numberOfTransitions);
For (Each Time Chunk)
  chunkPosition = representativeposition
  (positions, timeChunk);
Endfor
Function representativeposition
(positions, timeChunk)
{
  return mode(positions(timeChunk));
  # return median(positions(timeChunk));
  # return mean(positions(timeChunk));
}
```

As shown in the pseudo-code above, a predefined sleep period, or “sleep time,” can be divided into a positive integer “N” number of time chunks, with each time chunk having the same duration or time “length.” A representative position within the animation can be identified for each time chunk (e.g., using mode, median, mean etc.), to define a set of representative positions. The representative positions from the set of representative positions can then be combined into a single vector that describes the desired sequence of animation positions, optionally with overlaid text describing “insights,” as discussed below.

When generating the animation, system 100 may be configured to overlay time for each frame of the animation corresponding to a local time for that frame. In an example embodiment, the overlay time may be produced using the following pseudo-code:

For (Each Time Chunk)
Transition Video = Transition Videos
(where positions match the ones
in the Time Chunk)
OutputVideo = Concatenate
(OutputVideo, TransitionVideo)
Endfor
For(Each frame in OutputVideo)
TimeCode = getTimeCodeforFrame(frame)
Insight = getInsightFromInsightsList(frame)
OutputVideo = addOverlay(TimeCode)
OutputVideo =
addOverlayWithFadeInAndOut(Insight)
Endfor

As described herein with reference to FIG. 3A, system 100 can be configured to collect parameters 310 and generate insights 330. Insights 330 may be any suitable information for determining user 110's sleeping pattern. In an example embodiment, insights 330 may include a favorable sleeping position of user 110 (this information may be determined for a single sleeping session or may be determined by analyzing multiple sleeping sessions). For example, a text “Your favorite position is supine” (or any other position) may be presented to user 110 via interface 500.

Another example insight may include how often user 110 is switching positions. For example a text “You switched position 10 times” may be presented to user 110 via interface 500 to summarize all position transitions, as determined by system 100 (and shown via the generated animation).

In some cases, insight may include information about the respiratory quality. For example, an insight may inform user 110 that her/his respiratory quality degrades when she/he is in a particular position. For instance, the insight may include a text “Your respiratory quality degrades when you are in a prone position” (or any other position). In an example embodiment, respiratory quality may have an associated respiratory score. The respiratory score may be based on a blood oxygen level, or on a respiratory effort (as described above), or on both of these parameters. For example, the respiratory score may be an average (or weighted average, with appropriately selected weights) of these parameters. In cases when several (or all) of different positions of user 110's body have the same respiratory score, all of these positions can be shown for the same respiratory score. In an example embodiment, when several (or all) of different positions of user 110's body have the same respiratory score, a position in which user 110 spends most of the time may be shown via interface 500. Alternatively, or additionally, if one position has a particular low respiratory score, such a position may be shown.

In an example embodiment, insights 330 may include an indication of a position in which user 110 was particularly restful. For example, whether user 110 was restful may be determined by a pulse of user 110 and/or by a sleep stage of user 110. The indication may include a text, such as “You are most restful in supine position” (or any other position), and the text may be presented to user 110 via interface 500.

Additionally, or alternatively, insights 330 may include an indication of a position in which user 110 was particularly restless. For example, whether user 110 was restless may be determined by a pulse of user 110 and/or by a sleep stage of user 110. The indication may include a text, such as “You are most restless in prone position” (or any other position), and the text may be presented to user 110 via interface 500.

In some cases, a snoring insight may include information about whether user 110 snored in a particular position. For instance, the insight may include a text “You snored when you are in prone position” (or any other position). Additionally, the snoring insight may indicate snore parameters (e.g., a loudness of a snore, a pitch of the snore, a facial vibration amplitude due to the snore, and the like).

In various embodiments, any of the above examples of insights may be reported for a single sleeping session or may be evaluated and statistically analyzed for multiple sleeping sessions. For example, if user 110 was most restful in a prone position for the first and the third sleeping sessions, but was more restful in supine position for the second sleeping session, such information may be presented to user 110 via interface 500. Alternatively, user 110 may be informed that her/his most restful position is the prone position.

In some cases, user 110 may select the type of insight to be presented via interface 500. For example, user may choose insights from a list of possible available insights. In some cases, as described above, insights are configured to be strings containing one or more parameter fields that can be filled with particular numerical (alphanumerical, image, audio, graphical user interface) data. For example, a string for an insight may include “Your breathing quality was lowest in your [WORST_RESP_POSITION],” in which [WORST_RESP_POSITION] is a parameter field accepting a text value (e.g., “prone position”). In case above-mentioned insight cannot be clearly determined (e.g., if the breathing quality was identical across some/all of sleep positions) the above-mentioned insight may not be selected, and another insight may be selected. For example, another insight may be a string including “Your breathing quality was [RESP_QUALITY] through the night,” in which [RESP_QUALITY] is a parameter field accepting a numerical value corresponding, for example, to a respiratory score. It should be noted that any logic may be used to determine which (if any) of insights should be reported to user 110 based on user 110's sleep pattern (as well as user preferences, which user 110 may select via a preference/setting section of an application for displaying sleep parameters for user 110).

In various embodiments, interface 500 may present insights 330 using any suitable format. For example, insights may be presented via text of varying opacity (i.e., the text may be partially transparent). In some cases, text representing an example insight may fade to result in fade-in or fade-out effects.

In some cases, insights may include data (e.g., respiratory score) that may be generated using a computer model for determining such data. A computer model may, for example, include a machine-learning model. For instance, machine-learning model, such as a suitable neural network model (e.g., a convolutional neural network), or any other model (e.g., a decision tree model), may be used to determine the respiratory score from multiple parameters 310 collected by sensors of patches 111A-111F. In some cases, machine-learning models may be used to generate body position data, or any other useful data that may be used for generating insights (e.g., a machine-learning model may be used to divide a sleep session into time intervals corresponding to different sleeping positions).

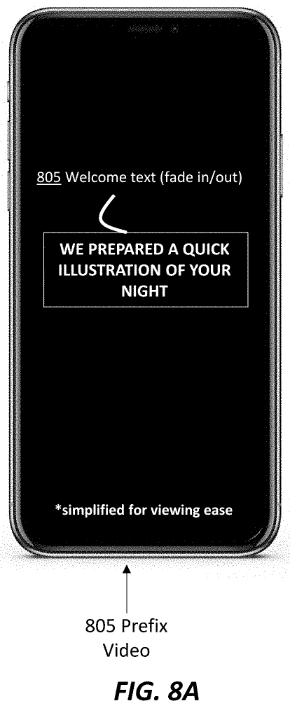

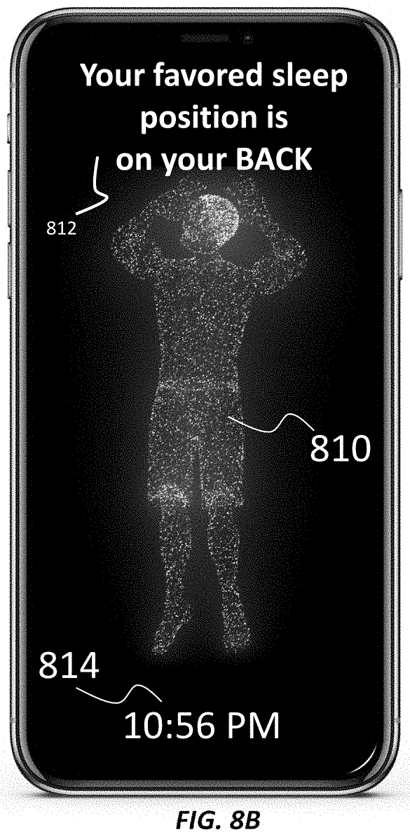

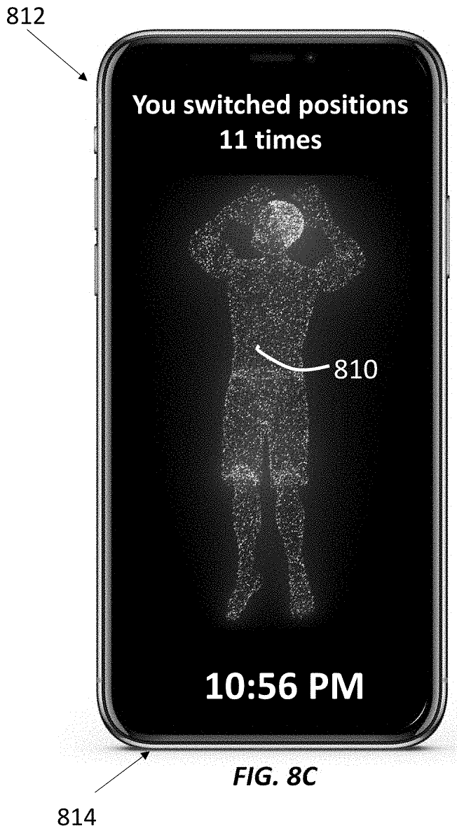

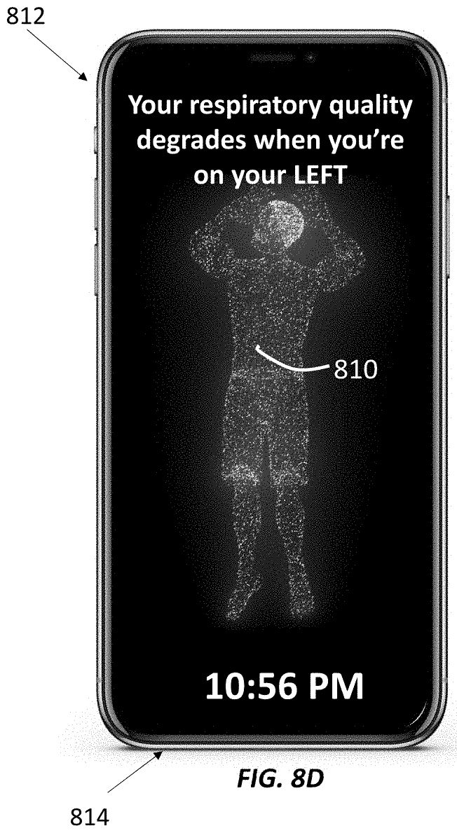

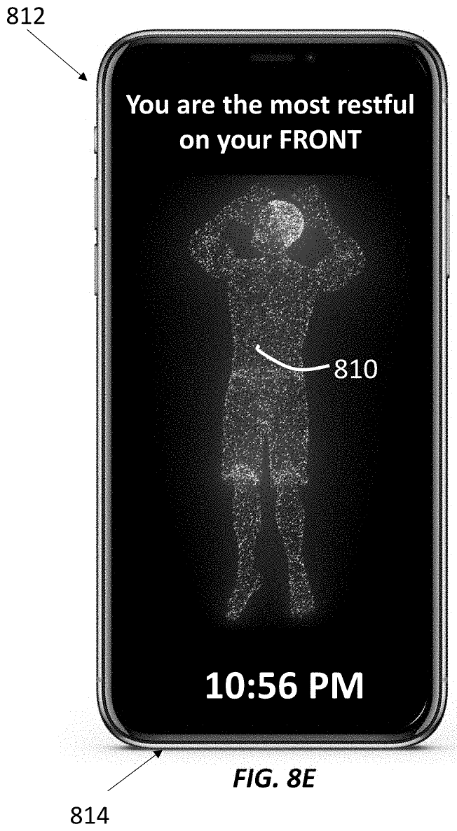

FIGS. 8A-8E show examples of displayed animation containing a prefix video and various insights. For instance, FIG. 8A shows a display containing prefix video 805 with introductory text (herein, also referred to as a welcome text) 805. In an example embodiment, a welcome text may be “WE PREPARED A QUICK ILLUSTRATION OF YOUR NIGHT.” The welcome text may be configured to fade-in and fade-out. FIG. 8B show an example insight text 812 and a position 810 of a body of user 110 at a time 814. In an example embodiment, insight text 812 includes “Your favored position is on your BACK” and time 814 is “10:56 PM.” FIG. 8C show another example insight text 812 and a position 810 of a body of user 110 at a time 814. In an example embodiment, insight text 812 includes “You switched positions 11 times.” FIG. 8D show another example insight text 812 and a position 810 of a body of user 110 at a time 814. In an example embodiment, insight text 812 includes “Your respiratory quality degrades when you're on your LEFT.” FIG. 8E show another example insight text 812 and a position 810 of a body of user 110 at a time 814. In an example embodiment, insight text 812 includes “You are the most restful on your FRONT.”

Table below further summarizes some of the insights that may be used. It should be noted that any other suitable insights may be used as well. The insights may be generated by processing parameters 310 such as a body movement, a body position, a sleep stage, a respiratory effort (the respiratory effort may be based on a measure of air flow (herein, referred to as a sum-flow)), a sum-flow, a heart rate, a blood oxygen level, audio related to snoring, body and room temperature, ambient lights, body and room humidity and bio-impedance of person's body (e.g., skin).

Favorite Position:
[FAV_POSITION] = The position with the most time during sleep.
Text: “Your favorite position was on your [FAV_POSITION]”
Position changes:
[POS_CHANGES] = number of position changes during sleep,
discounting short rapid transitions.
Text: “You switched positions [POS_CHANGES] times”
Respiratory Quality
[WORST_RESP_POSITION] = the position with the lowest Resp Quality score.
Text: “Your breathing quality was lowest on your [WORST_RESP_POSITION]″
If all positions scored the same:
[RESP_QUALITY] = The respiratory quality score of all positions
Text: ″Your breathing quality was [RESP_QUALITY] throughout the night”
Snoring
[WORST_SNORE_POSITION] = The position with the most snoring (in percentage).
Text: “You snored the most on your [WORST_SNORE_POSITION]”
If audio wasn't enabled, or snoring wasn't detected, or all positions
scored the same, the insight is not added to the video.

An example calculation of “Insights” is based on the gathered sensor data; and an example decision tree showing how to select which insights to present the user, as described above.

In various embodiments, a data received from sensors may be recorded by patches 111A-111F and may be transferred to a mobile computing device (e.g., compute device 113) which in turn saves it on a server. In an example embodiment the received data may not be analyzed (processed) and the data processing may be done on the server. The server may include a processing module for post-processing the received data and generate the appropriate outputs to be read by the WebViewer™ (or other apps) for producing insights for the session.

The processing module includes any suitable procedure or process for processing the raw data. The processed data may be placed on the cloud (e.g., AWS-S3). The desired outputs can be whatever the physiological or physical variable that are required for determining insights for the sleep study such as: breathing flow, intrathoracic pressure, Respiratory Inductive Plethysmography RIP signal, leg movement, artifact, SpO2 and cardiac pulsation. In some cases, the number of independent (or possible inter-linked) data that run through the postprocessing can be as many as the number of desired output variables. In some embodiments, all the output variables are extracted from the input measurements with an appropriate processing ranging from very simple filtering (like thorax and abdomen stretch signals) to much more complex (as in calculating SpO2). Some processing steps are in common between all the output variables including downloading data from the cloud, parsing and converting into processing format (CSV files), time correction and time alignment. However, some (or every) desired output variable has its own specific processing component as well.

In various embodiments, the sensory data may need to meet certain criteria in order for the processing module to be able to extract the desired output variables according to the standard requirements. Since the input data are collected from various sensors such as accelerometers, stretch sensors, light sensors, such as red or infrared light sensors, as well as temperature sensors, the constraints can be associated with either all the sensors, or specific sensors. An example table, bellow, summarizes possible constrains that need to be satisfied.

| No. | Constraint | Sensor Affected |
|---|---|---|
| 1 | Patches need to stay in close and robust | All sensors |
| | attachment to the body skin | |
| 2 | No part of patch might be peeled or taken | All sensors |
| | off during the data recording | |
| 3 | The reflectance light data is required to be | Pulse Oximeter |
| | off saturation | (Pox) |

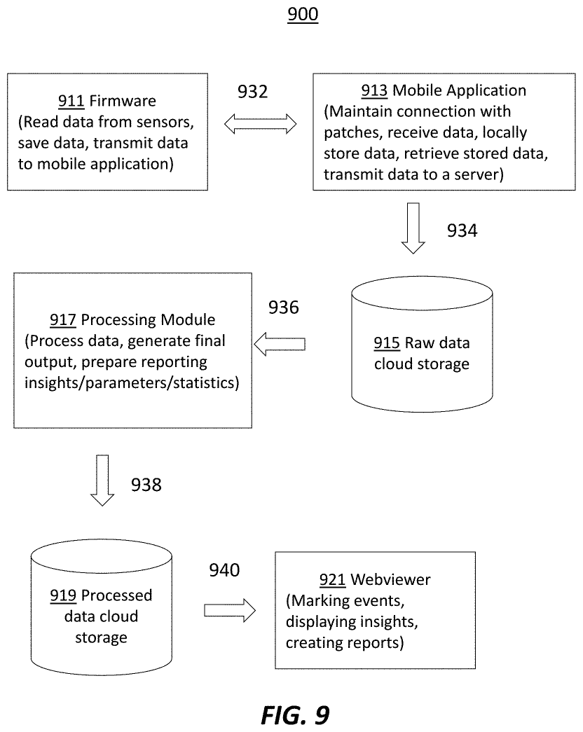

An example diagram 900 for processing sensor data for reporting processed data is shown in FIG. 9. In an example embodiment, a firmware component 911 of a patch (firmware 911 being an application residing in a memory of the patch) may be configured to instruct a processing device of the patch to read data from the sensors of the patch, save the obtained sensor data in a local memory associated with the patch, and transmit the saved data to an application of a mobile device (e.g., compute device 113). Firmware 911 is configured to send and receive information with a mobile application 913 of compute device 113 as indicated by arrow 932. Mobile application 913 is configured to maintain connection with patches 111A-111F, receive data from firmware 911, locally store data, retrieve the locally stored data and transmit the data to a server as indicated by arrow 934. The server may store the raw data at a cloud storage 915. Further, the server is configured to use processing module 917 to process raw data, generate final output and prepare reporting insights, and/or parameters, and/or statistics. The server is configured to transmit the prepared data to a processed data cloud storage 919. Further, the processed data may be transmitted to the WebViewer™, as indicated by arrow 940 for marking events associated with a sleep session of user 110, displaying insights, and creating any suitable reports displaying information associated with the sleep session.

In an example embodiment, during the postprocessing, data from each patch is processed separately, and once they are processed, their results are merged at a patch merging stage. In various embodiments, processing module 917 may process raw data as the raw data is uploaded to raw data cloud storage 915. The processed data then may be transmitted to a compute device 113 for displaying the results associated with the processed data in real time.

In an example embodiment, after processing module 917 processes data for individual patches, data from different patches is aligned in time, and various time-based characteristics are computed based on the time-aligned data. An example of aligning of data from a first and a second patch may be as follows. The first patch may include a sensor that determines a blood oxygen levels at times T1 and T3, while the second patch may include a sensor for determining accelerometer data at time T2, T1<T2<T3. In an example embodiment, to align blood oxygen levels with accelerometer data at time T2, processing module 917 may be configured to interpolate blood oxygen levels at time T2 (e.g., using a spline interpolation, or any other suitable interpolation technique).

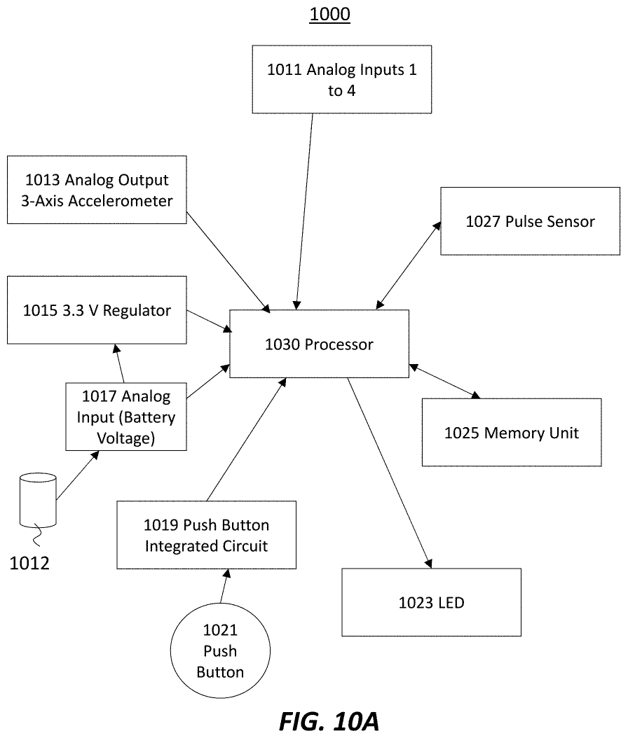

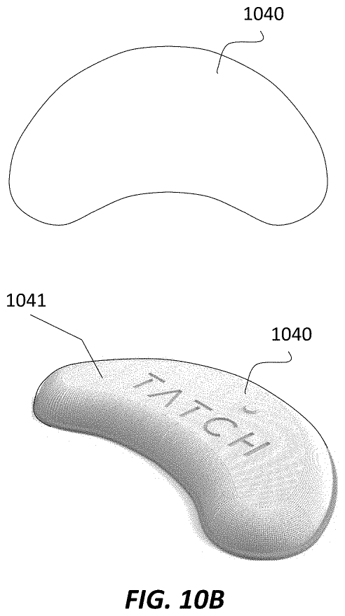

FIG. 10A shows an example diagram 1000 of an electrical hardware of a patch (e.g., patch 111). In an example embodiment, the hardware may include a printed circuit board (PCB) of approximately 1 inch by linch square. The electrical hardware may be designed to provide a small, low-power, wirelessly connected wearable patch to provide reliable physiological measurement data to a smartphone or tablet device. Diagram 1000 includes a processor 1030 (e.g., Nordic NRF52832 BLE Module) for processing data from analog inputs 1011, a 3-axis accelerometer sensor 1013 (e.g., ADXL335BCPZ), and a pulse sensor 1027 (e.g., MAX30101). It should be noted that various other sensors may be used. Analog inputs 1011 may include data recorded by flexible and/or stretch sensors, body temperature data, body humidity data and the like. Further, the electrical hardware may include a battery 1012 (e.g., a 7V lithium ion battery), a voltage regulator (e.g., a 3.3 V regulator for converting the voltage of the battery to an acceptable voltage used by processor 1030), an indicator of battery voltage 1017, a push button 1021 for controlling various aspects of processor 1030 (e.g., for resetting processor 1030), and an integrated circuit 1019 (e.g., STM6601AQ2BDM6F) associated with push button 1021. Further, the electrical hardware includes a memory unit 1025 (e.g., a flash memory with a corresponding integrated circuit, such as W25No1GVZE) and a light emitting diode 1023 indicating a status of patch 111 (e.g., whether patch 111 is on, and/or if it is processing and/or transmitting data). In an example embodiment, processor 1030 may include a Bluetooth module or a Wireless module for sending/receiving data from compute device 113. Additionally, the electrical hardware may include an external crystal oscillator for timing accuracy. In various embodiments, the PCB may be made from either rigid or flexible material to improve ergonomics and sensor performance. In some cases, the PCB may include a battery recharging circuit and external port (e.g., micro USB). Additionally or alternatively, the PCB may be configured to be charged wirelessly.

FIG. 10B shows a top and isometric view of patch 111. In an example embodiment a top surface 1041 of patch 1040 is made from a fabric (e.g., a stretchable fabric), flexible rubber, flexible plastic and the like, and a bottom surface of patch 111 is made from a highly stretchable double-sided medically adhesive material that may be applied directly to a skin of user 110. In an example embodiment, patch 111 may have top fabric a middle fabric and a bottom fabric. Top fabric may be configured to bring softness and comfort, a middle fabric may work as a filled layer which can reduce the bumps caused by the PCB and battery 1012. Further, the middle fabric may add more comfort to patch 111. In an example embodiment, top fabric layer is configured to seal the edges of patch 111 by directly attaching to bottom fabric via a fabric-to-fabric suitable connection.

In various embodiments, when system 100 generates a sleep report, different parameters may be processed, and generated output may depend on information obtained from observed data. In an example embodiment a logic rules determine which data will be displayed, and how data will be processed and shown. The logic rules may include reporting a body position of user 110 when user 110 is sleeping (i.e., excluding wake times, and non-sleep positions such as upright positions) For instance, system 100 may evaluate number of upright positions and a duration of time user 110 spent in the upright position for a given time interval, and based on the evaluation, determine whether user 110 is sleeping or awake.

Further, system 100 may determine per-position scores (e.g., various sleep data for user related to a particular position). In an example embodiment, if user 110 spends less than 45 minutes in a particular position, system 100 may be configured to report that there is insufficient respiratory data. Alternatively, system 100 may collect various respiratory parameters, as described above, associated with a sleep session of user 110. Further, if a snoring is detected, system 100 may be configured to determine which body positions resulted in snoring.

Further, as described above, system 100 may collect data to report various insights, such as time lapse insights. For example, if all positions have equal respiratory quality, system 100 may not report a “worst” position, and report “your respiratory quality was good throughout the night.” If all positions have equal snore quality, system 100 may not report a “worst” position. As described above, system 100 may ignore upright/unknown positions. Further, system 100 may report various data associated with snoring. In some cases, system 100 may be configured not to report snoring if a total snoring time is less than 15 minutes. For reporting respiration for a particular position of user 110's body, system 100, may use weighted average respiration score averaged over a duration of time user 110 spent in that particular position.

In some cases, system 100 may be configured to generate an overall report related to a sleep session of user 110. In some cases, a report may be submitted to a medical professional for analysis. In case the sleeping session is less than 4 hours, the report may not be submitted. Further, the report may not be submitted if there is a low confidence in data obtained by sensors (e.g., if a confidence in data is less than 75% based on how the obtained data is compared (i.e., calibrated) with a historical data for user 110. For instance, if it is historically recorded that user 110 is usually relaxed in a prone position and is uncomfortable sleeping on her side, a confidence in data indicating that user is comfortable on her side may be low.

In various embodiments, some of sleep parameters (e.g., parameters 310) may be flagged if they are outside of normal ranges or if they are unusual or inconsistent with other parameters. For example, if system 100 is unable to determine (or has low confidence in) an orientation of a patch (e.g., patch 111), the inability of system 100 to determine the orientation may be flagged (e.g., if confidence of determination of orientation is less than 0.5). System 100 may flag a poor respiratory signal quality if the signal quality is unclear more than twenty percent of the sleep time. System 100 may indicate (flag) if a position of a body of user 110 has changed more than six times in an hour (the position is determined to be changed based on an associated metric as discussed above). Further, system 100 may produce an indication that user 110 slept more than ten hours, or snored for more than 85 percent of the sleep time, etc.

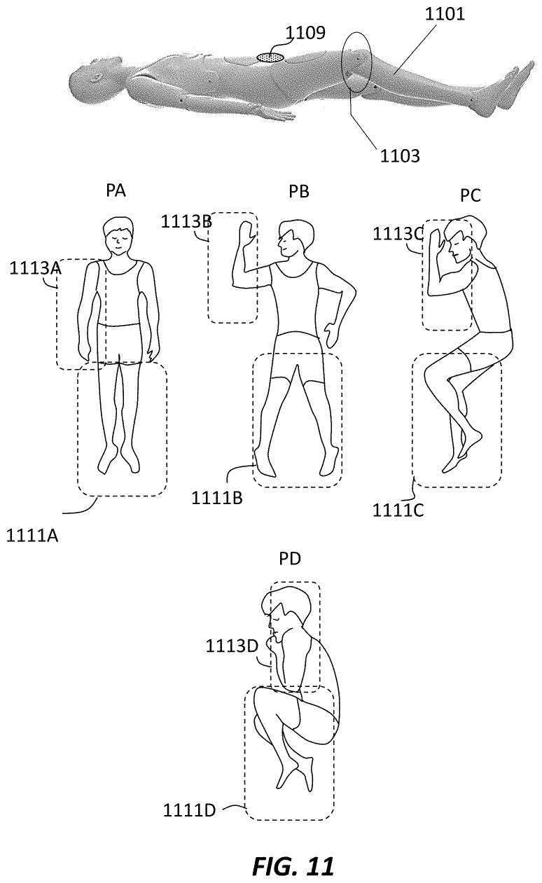

In various embodiments, the placement of patches 111A-111F (shown in FIG. 1) may be optimized based on a number of available patches, as well as based on various sleep parameters 310 of user 110 that need to be tracked. For example, if user 110 has a tendency to swing her/his arms during sleep and such motions need to be documented, patches may be placed on the arms of user 110. An example optimization procedure may include one or more computer simulations. For example, a simulated body 1101, as shown in FIG. 11, may be a mechanical model of a person's body comprising various body parts (e.g., shoulders, upper arms, lower arms, wrists, thighs, etc.) connected by joints, such as joint 1103. During a simulation process, simulated patches (herein, also referred to as virtual patches) may be placed at the first locations of the mechanical model, and body positions may be analyzed. If data obtained from the virtual patches results in a set of sensed body positions that match an actual set of body positions, then the locations of virtual patches are accepted. If, however, data obtained from the virtual patches does not result in a set of sensed body positions that match the actual set of body positions, then the locations of virtual patches may be altered, and the simulation may be repeated. In some cases, the simulation may determine an adequate number of virtual patches needed for resolving a position of a body. For instance, if only one virtual patch 1109 is used, then such a patch may differentiate between the general orientations of a body of user 110 (e.g., virtual patch 1109 mat differentiate between positions PA, PB, and PC, but may not differentiate between positions PC and PD). Further, virtual patch 1109 may not be able to determine positions of user limbs as shown by regions 1111A-1111D, and 1113A-1113D). In order to resolve a position of a user with higher accuracy, patches for arms and legs may be needed. In an example embodiment, a specific location for these patches may be determined via the computer simulation described above.

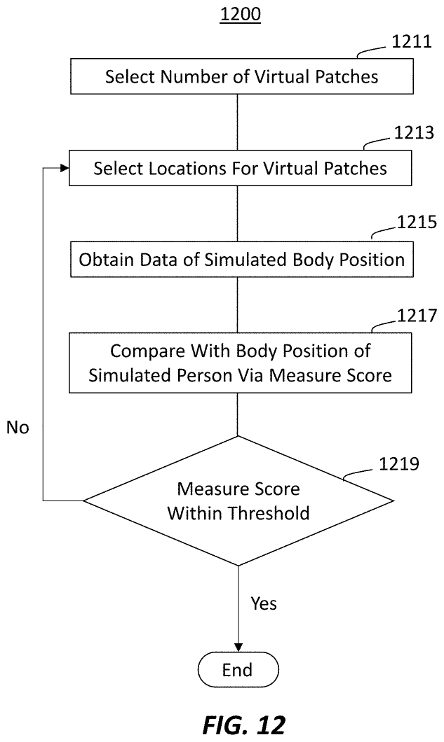

FIG. 12 shows an example process 1200 for determining the location of virtual patches in order to obtain an accurate determination of a position of a body of user 110, given a selected number of virtual patches. Process 1200 may be used for the computer simulations described above. At step 1211 a selected number of virtual patches may be used for the computer simulation. At step 1213, locations for virtual patches may be selected. At step 1215, data from virtual patches is obtained to determine sensed body positions, and at step 1217, the sensed body positions are compared with the actual body positions via, for example, measure scores calculations. At step 1219, the measure scores are compared within predetermined threshold values. If all of the measure scores (a measure score is calculated for a given position of the mechanical body model and illustrates a difference between the sensed body position and the actual body position) are within the predetermined threshold value (step 1219, Yes) placements of virtual patches are accepted. Alternatively, if at least one of the measure scores is not within the predetermined threshold value (step 1219, No), process 1200 proceed to step 1213 and new locations of virtual patches are selected. It should be noted that new locations of virtual patches may be determined using any suitable iterative process for minimizing measure scores (e.g., gradient descent algorithm, conjugate gradient algorithm, and the like).

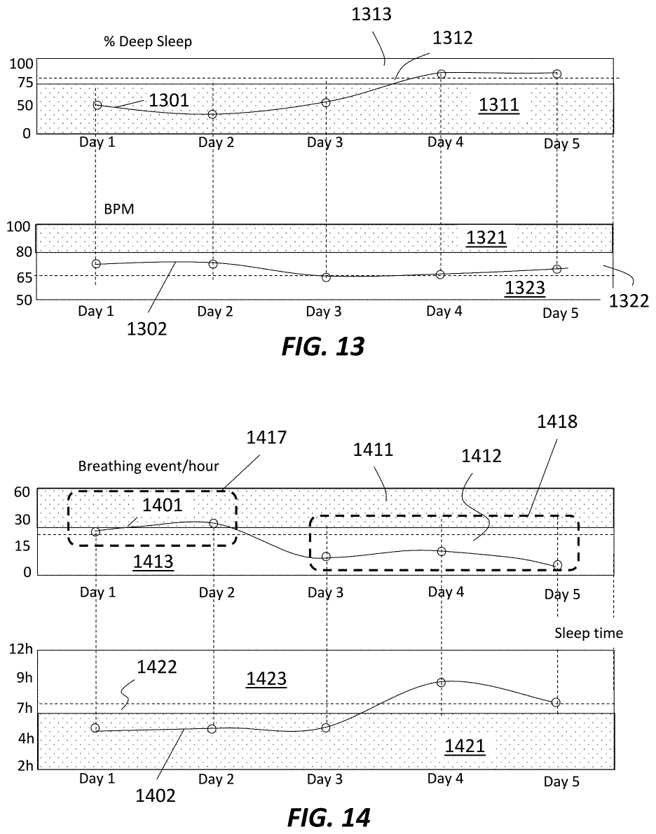

As described above, data (e.g., respiratory parameters or any other parameters related to a user's sleep) collected for each night of sleep may be aggregated to present sleep trends over time. FIG. 13 shows an example plot 1301 depicting a percentage of time a person spent in a deep sleep (e.g., NREM N3) during a nightly sleep and a time-correlated plot 1302 depicting heart rate in beats per minute (BPM) (the heart rate may be averaged over a few hours of sleep or over a night of sleep). The plot 1301 is subdivided into an interval 1313 corresponding to a desired sleep quality (e.g., a restful deep sleep), an interval 1311 corresponding to relatively restless sleep, and an interval 1312 corresponding to sleep quality that is transitional between the restless stage (interval 1311) and restful stage (interval 1313). The plot 1301 represents a sleep trend of a person determined over a time interval of five days. For example, in the first 3 days (day 1 through day 3) it shows that the person experienced relatively restless sleep (e.g., less than 50% of the time was spent in a deep sleep), while last two days shows that the person experienced restful sleep (e.g., more than 50% of time was spend in a deep sleep).

As can be observed in FIG. 13, the trend shown by plot 1301 correlates, to a degree, with a trend shown by plot 1302. For instance, in the first 3 days (day 1 through day 2) it shows that the person's nightly heart rate was averaging about 70 beats per minute, while on day 3 and day 4, the heart rate of the person was lower—about 65 beats per minute. Note that on day 3, the person experienced a lower heart rate while still exhibiting relatively restless sleep (e.g., on day 3 the person spent only about 50% of the time in a deep sleep). Thus, plots 1301 and 1302 are not in perfect correlation, and other parameters may be considered to determine factors affecting the sleep quality of a person. The plot 1302 is subdivided into an interval 1323 corresponding to a desired heart rate (e.g., a restful heart rate), an interval 1321 corresponding to a relatively restless heart rate, and an interval 1322 corresponding to a heart rate that is transitional between the restless heart rate (interval 1321) and restful heart rate (interval 1323).

It should be noted that the trends represented by plots 1301 and 1302 are only illustrative, and any other suitable trends may be collected over a course of several seconds, minutes, hours, days, weeks, months, years, and the like. While one parameter (heart rate) is shown in FIG. 13, any other suitable parameter or a group of parameters may be correlated with a quality of the sleep to further understand the sleeping trends and factors affecting the person's sleep.

Further, in some cases, the trends may be collected and analyzed for a group (or groups) of individuals that are unified by particular events, diseases, location, food consumption, drug consumption, lifestyle, sexual orientation, and the like (e.g., the trend may be collected and analyzed for middle-aged veterans of a Gulf War diagnosed with a PTSD).

FIG. 14 shows other examples of trends that may be observed and analyzed. For example, plot 1401 shows a number of breathing events per hour (e.g., breathing disruptions which may include brief breathing interruptions, or any other breathing disruptions described above, such as apnea, hypopnea, eupnea, and the like). The plot 1401 is subdivided into an interval 1413 corresponding to a low number of breathing disruptions (e.g., less than 15 breathing disruptions per hour), an interval 1411 corresponding to a relatively high number of breathing disruptions (e.g., more than about 20 breathing disruption per hour), and an interval 1422 corresponding to a heart rate that is transitional between the interval 1411 and the interval 1413.

FIG. 14 also shows a time-correlated plot 1402 depicting sleep time of a person during a night. The ordinate of a graph corresponding to plot 1402 is subdivided into an interval 1423 corresponding to a restful night in which a person slept more than 7 hours of sleep, an interval 1421 corresponding to a relatively restless night (e.g., a night in which the person slept less than 7 hours) and an interval 1422 corresponding to night that is transitional between the restless night (interval 1421) and restful night (interval 1423). The plot 1402 represents a sleep trend for a person determined over a time interval of five days. For example, in the first 3 days (day 1 through day 3) it shows that the person experienced relatively restless nights (e.g., on those nights the person slept only about 4 hours), while last two days shows the person experiencing restful nights (e.g., the person slept more than 7 hours on those nights).

As shown in an upper graph of FIG. 14 at region 1417, corresponding to the first two days, a person did not receive any therapy for treating breathing disruptions during a “baseline” period (e.g., the person did not use an oral appliance or a continuous positive airway pressure (CPAP) device), while in the last three days, as indicated by region 1418, the person used sleep therapy (e.g., an oral appliance or a CPAP device). Plots 1401 and 1402 indicate a clear trend that the oral appliance was effective in improving the quality of sleep of the person.

In some embodiments, a system for monitoring a sleep of a user includes a plurality of patches for placement adjacent to a surface of a body of a user, a processor, and a data communication system. Each patch from the plurality of patches includes at least one sensor. The data communication system transmits positional data generated by the plurality of sensors, including orientation data and motion data, to the processor. The processing of the positional data includes determining a first position of the body of the user at a first time and a first image based on the first position of the body of the user at the first time. A change in position of the body of the user is detected based on a measure function and a threshold value. In response to detecting the change in position of the body of the user, a second position of the body of the user is determined, at a second time subsequent to the first time, and a second image is determined based on the second position of the body of the user at the second time. Based on the first image and the second image, an animation of a movement of the body from the first position to the second position is generated. The animation can be an accelerated time-lapse animation. For example, positional data may be generated by the plurality of sensors over the course of 8 hours of sleep, whereas the animation may present a progression of positions (or movement of the user) detected within that 8 hour period within a comparatively brief time period (e.g., about 2 seconds, about 5 seconds, about 30 seconds, about 1 minute, etc.).

In some implementations, at least one sensor from the plurality of sensors includes at least one of: one or more accelerometers or one or more micro-electromechanical gyroscopes.

In some implementations, the processor is further configured to determine whether the user is in a vertical position, a seated position, or a horizontal position.

In some implementations, the processor is configured not to process positional data when the user is in the vertical position.

In some implementations, each patch from the plurality of patches further includes a pressure sensor for generating pressure data and/or a pulse sensor for generating pulse data.

In some implementations, each patch from the plurality of patches further includes a sensor for generating data related to a respiratory effort.

In some implementations, each patch from the plurality of patches further includes an oximeter for generating blood oxygen level data and/or a temperature sensor for detecting a body temperature of the user.

In some implementations, the processor is configured to determine whether the user is sleeping based on (1) the determination as to whether the user is in a vertical position, a seated position, or a horizontal position, and (2) at least one of respiratory effort or pulse data determined for a first predefined time period. The processor can further be configured to determine whether the user is sleeping further based on a change in respiratory effort or a change in pulse data during a second predefined period of time subsequent to the first predefined time period. The processor may also be configured not to process positional data when the processor determines that the user is not sleeping. In other implementations, the processor can be configured to determine whether the user is sleeping based on actigraphy. In still other implementations, the processor can be configured to determine whether the user is sleeping based on actigraphy and based on heart rate data associated with the user. In still other implementations, the processor can be configured to determine whether the user is sleeping based on actigraphy and based on respiratory data associated with the user.

In some implementations, each patch from the plurality of patches further includes one of an audio sensor, a nasal pressure sensor, or a vibrational sensor.

In some implementations, the animation includes a representation of at least one of: pressure data, respiratory effort, pulse data, blood oxygen level data, temperature data, or a snoring condition of the user.

In some implementations, the processor is configured to estimate a sleep stage of the user based on movement of the body of the user.

In some implementations, the processor is further configured to detect a significant change in sensed data, the sensed data including one of pressure data, respiratory effort, respiratory flow (e.g., nasal airflow), respiratory pressure (e.g., nasal air pressure), pulse data, blood oxygen level data, temperature data, or snoring data, based on a predefined threshold.

In some implementations, the system also includes a display, and the processor is further configured to present the animation via the display.

In some embodiments, a method for monitoring a sleep of a user includes positioning a plurality of patches adjacent to a surface of a body of a user. Each patch from the plurality of patches includes an associated sensor from a plurality of sensors. The method also includes causing positional data generated by the plurality of sensors to be transmitted to a processor, the positional data including orientation data and motion data. The processing of the positional data via the processor includes determining a first position of the body of the user at a first time, determining a first image based on the first position of the body of the user at the first time, and detecting a change in position of the body of the user based on a measure function and a threshold value. In response to detecting the change in position of the body of the user, a second position of the body of the user at a second time subsequent to the first time is determined. A second image is determined based on the second position of the body of the user at the second time. Based on the first image and the second image, an animation is generated of a movement of the body from the first position of the body of the user at the first time to the second position of the body of the user at the second time.

In some embodiments, a non-transitory computer readable medium stores instructions that, when executed by a processor, cause the processor to perform operations including determining a first position of the body of the user at a first time, determining a first image based on the first position of the body of the user at the first time, and detecting a change in position of the body of the user based on a measure function and a threshold value. The operations also include, in response to detecting the change in position of the body of the user, determining a second position of the body of the user at a second time subsequent to the first time, and determining a second image based on the second position of the body of the user at the second time. An animation of a movement of the body from the first position of the body of the user at the first time to the second position of the body of the user at the second time is generated based on the first image and the second image.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments can be implemented in any of numerous ways. For example, embodiments of the present technology may be implemented using a combination of hardware, and software (or firmware). When implemented in firmware and/or software, the firmware and/or software code can be executed on any suitable processor or collection of logic components, whether provided in a single device or distributed among multiple devices.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The terms “substantially,” “approximately,” and “about” used throughout this Specification and the claims generally mean plus or minus 10% of the value stated, e.g., about 100 would include 90 to 110.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A system for monitoring a sleep of a user, the system comprising:
a plurality of patches, each patch from the plurality of patches including an associated sensor from a plurality of sensors, each patch from the plurality of patches configured to be positioned adjacent to a surface of a body of a user;
a processor configured to process positional data generated by the plurality of sensors, the positional data including orientation data and motion data; and
a data communication system configured to transmit the positional data to the processor;
the processing of the positional data including:
determining a first position of the body of the user at a first time;
determining a first image based on the first position of the body of the user at the first time;
detecting a change in position of the body of the user based on a measure function and a threshold value;
in response to detecting the change in position of the body of the user:
determining a second position of the body of the user at a second time subsequent to the first time, and
determining a second image based on the second position of the body of the user at the second time; and
generating, based on the first image and the second image, an animation of a movement of the body from the first position of the body of the user at the first time to the second position of the body of the user at the second time.

2. The system of claim 1, wherein at least one sensor from the plurality of sensors includes an accelerometer.

3. The system of claim 1, wherein at least one sensor from the plurality of sensors includes a micro-electromechanical gyroscope.

4. The system of claim 1, wherein the processor is further configured to determine whether the user is in a vertical position, a seated position, or a horizontal position.

5. The system of claim 4, wherein the processor is configured not to process positional data when the user is in the vertical position.

6. The system of claim 1, wherein each patch from the plurality of patches further includes a pressure sensor for generating pressure data.

7. The system of claim 1, wherein each patch from the plurality of patches further includes a sensor for generating data related to a respiratory effort.

8. The system of claim 1, wherein each patch from the plurality of patches further includes a pulse sensor for generating pulse data.

9. The system of claim 1, wherein each patch from the plurality of patches further includes an oximeter for generating blood oxygen level data.

10. The system of claim 1, wherein each patch from the plurality of patches further includes a temperature sensor for detecting a body temperature of the user.

11. The system of claim 1, wherein the processor is configured to determine whether the user is sleeping based on (1) the determination as to whether the user is in a vertical position, a seated position, or a horizontal position, and (2) at least one of respiratory effort or pulse data determined for a first predefined time period.

12. The system of claim 11, wherein the processor is further configured to determine whether the user is sleeping further based on a change in respiratory effort or a change in pulse data during a second predefined period of time subsequent to the first predefined time period.

13. The system of claim 11, wherein the processor is configured not to process positional data when the processor determines that the user is not sleeping.

14. The system of claim 1, wherein each patch from the plurality of patches further includes one of an audio sensor, a nasal pressure sensor, or a vibrational sensor.

15. The system of claim 1, wherein the animation includes a representation of at least one of: pressure data, respiratory effort, pulse data, blood oxygen level data, temperature data, or a snoring condition of the user.

16. The system of claim 1, wherein the processor is configured to estimate a sleep stage of the user based on movement of the body of the user.

17. The system of claim 1, wherein the processor is further configured to detect a significant change in sensed data, the sensed data including one of pressure data, respiratory effort, pulse data, blood oxygen level data, temperature data, or snoring data, based on a predefined threshold.

18. The system of claim 1, further comprising a display, and wherein the processor is further configured to present the animation via the display.

19. The system of claim 1, wherein the animation is an accelerated time-lapse animation.

20. A method for monitoring a sleep of a user, the method comprising:
positioning a plurality of patches adjacent to a surface of a body of a user, each patch from the plurality of patches including an associated sensor from a plurality of sensors;
causing positional data generated by the plurality of sensors to be transmitted to a processor, the positional data including orientation data and motion data;
processing the positional data via the processor by:
determining a first position of the body of the user at a first time;
determining a first image based on the first position of the body of the user at the first time;
detecting a change in position of the body of the user based on a measure function and a threshold value;
in response to detecting the change in position of the body of the user, determining a second position of the body of the user at a second time subsequent to the first time;
determining a second image based on the second position of the body of the user at the second time; and
generating, based on the first image and the second image, an animation of a movement of the body from the first position of the body of the user at the first time to the second position of the body of the user at the second time.

21. A non-transitory computer readable medium storing instructions that, when executed by a processor, cause the processor to perform operations comprising:
determining a first position of the body of the user at a first time;
determining a first image based on the first position of the body of the user at the first time;
detecting a change in position of the body of the user based on a measure function and a threshold value;
in response to detecting the change in position of the body of the user, determining a second position of the body of the user at a second time subsequent to the first time;
determining a second image based on the second position of the body of the user at the second time; and
generating, based on the first image and the second image, an animation of a movement of the body from the first position of the body of the user at the first time to the second position of the body of the user at the second time.
